SEQUENCE LISTING (156 sequences)

SEQ ID NO: 1 - DNA, 110 bp, synthetic construct
  source 1..110: mol_type = other DNA
ccatctattattttattaatttatctcctccgaactgcctagtagtagcactgatactgcaaatacacagatatttcaaatgggcatttaaaagggcagtttccatgggc

SEQ ID NO: 2 - DNA, 110 bp, synthetic construct
  source 1..110: mol_type = other DNA
atctcctccgaactgcctagtagtagcactgatactgcaaatacacagatatttcaaatgggcatttaaaagggcagtttccatgggctcattttgagttgcaacagagg

SEQ ID NO: 3 - DNA, 110 bp, synthetic construct
  source 1..110: mol_type = other DNA
gtagcactgatactgcaaatacacagatatttcaaatgggcatttaaaagggcagtttccatgggctcattttgagttgcaacagaggtttatcagatatatcaaggttt

SEQ ID NO: 4 - DNA, 110 bp, synthetic construct
  source 1..110: mol_type = other DNA
acagatatttcaaatgggcatttaaaagggcagtttccatgggctcattttgagttgcaacagaggtttatcagatatatcaaggttttaccctttctttaaggatagag

SEQ ID NO: 5 - DNA, 110 bp, synthetic construct
  source 1..110: mol_type = other DNA
taaaagggcagtttccatgggctcattttgagttgcaacagaggtttatcagatatatcaaggttttaccctttctttaaggatagaggccaaatctagtattgtttata

SEQ ID NO: 6 - DNA, 110 bp, synthetic construct
  source 1..110: mol_type = other DNA
tgagagctgtttagtaataatgcagttttcttgaggtctgtttattttatagttttttaaaaaaccttcagttctttcggccagcgtgtgtgtattatctgtgcattaat

SEQ ID NO: 7 - DNA, 110 bp, synthetic construct
  source 1..110: mol_type = other DNA
cagttttcttgaggtctgtttattttatagttttttaaaaaaccttcagttctttcggccagcgtgtgtgtattatctgtgcattaatggtcctcatctgactactgcat

SEQ ID NO: 8 - DNA, 110 bp, synthetic construct
  source 1..110: mol_type = other DNA
ttttatagttttttaaaaaaccttcagttctttcggccagcgtgtgtgtattatctgtgcattaatggtcctcatctgactactgcattggttcatgtttttctgcatgg

SEQ ID NO: 9 - DNA, 110 bp, synthetic construct
  source 1..110: mol_type = other DNA
ttcagttctttcggccagcgtgtgtgtattatctgtgcattaatggtcctcatctgactactgcattggttcatgtttttctgcatggattgacataaaaccattactaa

SEQ ID NO: 10 - DNA, 110 bp, synthetic construct
  source 1..110: mol_type = other DNA
tgtgtattatctgtgcattaatggtcctcatctgactactgcattggttcatgtttttctgcatggattgacataaaaccattactaaaatttggcacctatgagatatc

SEQ ID NO: 11 - DNA, 110 bp, synthetic construct
  source 1..110: mol_type = other DNA
cgtgaaagggccatattaatcattaggcaagacaaaccacggaaattcctattaagcaacatctcagttctccccagaaagtccccctaaggctacttattcactcttta

SEQ ID NO: 12 - DNA, 110 bp, synthetic construct
  source 1..110: mol_type = other DNA
ttaggcaagacaaaccacggaaattcctattaagcaacatctcagttctccccagaaagtccccctaaggctacttattcactctttaatcttgactacacataggctcc

SEQ ID NO: 13 - DNA, 110 bp, synthetic construct
  source 1..110: mol_type = other DNA
attcctattaagcaacatctcagttctccccagaaagtccccctaaggctacttattcactctttaatcttgactacacataggctccagttttccctggctctggatct

SEQ ID NO: 14 - DNA, 110 bp, synthetic construct
  source 1..110: mol_type = other DNA
gttctccccagaaagtccccctaaggctacttattcactctttaatcttgactacacataggctccagttttccctggctctggatctcactgcattgtcccttggtcct

SEQ ID NO: 15 - DNA, 110 bp, synthetic construct
  source 1..110: mol_type = other DNA
aaggctacttattcactctttaatcttgactacacataggctccagttttccctggctctggatctcactgcattgtcccttggtcctgctctcccccttacctcctgca

SEQ ID NO: 16 - DNA, 110 bp, synthetic construct
  source 1..110: mol_type = other DNA
aaaaaagaaacacatgcggacatagtccacacactcacagacacagataccatgataaattccagaccacaagacagactgggaaagtctccctctctcctttttttttt

SEQ ID NO: 17 - DNA, 110 bp, synthetic construct
  source 1..110: mol_type = other DNA
tagtccacacactcacagacacagataccatgataaattccagaccacaagacagactgggaaagtctccctctctcctttttttttttttaatgaattttcttgagatt

SEQ ID NO: 18 - DNA, 110 bp, synthetic construct
  source 1..110: mol_type = other DNA
agataccatgataaattccagaccacaagacagactgggaaagtctccctctctcctttttttttttttaatgaattttcttgagattagcttcagcattttagccagta

SEQ ID NO: 19 - DNA, 110 bp, synthetic construct
  source 1..110: mol_type = other DNA
ccacaagacagactgggaaagtctccctctctcctttttttttttttaatgaattttcttgagattagcttcagcattttagccagtattttaaggactaacagacttat

SEQ ID NO: 20 - DNA, 110 bp, synthetic construct
  source 1..110: mol_type = other DNA
ctccctctctcctttttttttttttaatgaattttcttgagattagcttcagcattttagccagtattttaaggactaacagacttatgagttaccatcattggcatcta

SEQ ID NO: 21 - DNA, 110 bp, synthetic construct
  source 1..110: mol_type = other DNA
ttttttctctgtgaatagcttttaactaaaaactgtcagttacccagtgtttctcaacagagtactaatggcatttgggttgacagttcttaattgggcgggctctcttg

SEQ ID NO: 22 - DNA, 110 bp, synthetic construct
  source 1..110: mol_type = other DNA
taactaaaaactgtcagttacccagtgtttctcaacagagtactaatggcatttgggttgacagttcttaattgggcgggctctcttgttcccactaaatgccagagcgt

SEQ ID NO: 23 - DNA, 110 bp, synthetic construct
  source 1..110: mol_type = other DNA
cagtgtttctcaacagagtactaatggcatttgggttgacagttcttaattgggcgggctctcttgttcccactaaatgccagagcgtgacaaaaacgctgtcatgcaca

SEQ ID NO: 24 - DNA, 110 bp, synthetic construct
  source 1..110: mol_type = other DNA
aatggcatttgggttgacagttcttaattgggcgggctctcttgttcccactaaatgccagagcgtgacaaaaacgctgtcatgcacacattttaaaatactccttcatt

SEQ ID NO: 25 - DNA, 110 bp, synthetic construct
  source 1..110: mol_type = other DNA
cttaattgggcgggctctcttgttcccactaaatgccagagcgtgacaaaaacgctgtcatgcacacattttaaaatactccttcattggatgctgcccccctcccccgc

SEQ ID NO: 26 - DNA, 110 bp, synthetic construct
  source 1..110: mol_type = other DNA
ttatgatgtgccaggcctgatgaacacaccctcattaattcctcactaccatcttatgaggtagatgtcacctttataccatttaacaaataaaaaaacgcaatcttgga

SEQ ID NO: 27 - DNA, 110 bp, synthetic construct
  source 1..110: mol_type = other DNA
aacacaccctcattaattcctcactaccatcttatgaggtagatgtcacctttataccatttaacaaataaaaaaacgcaatcttggaaagcttagtacttccctgtagc

SEQ ID NO: 28 - DNA, 110 bp, synthetic construct
  source 1..110: mol_type = other DNA
actaccatcttatgaggtagatgtcacctttataccatttaacaaataaaaaaacgcaatcttggaaagcttagtacttccctgtagctcagatggtgaagaatctgcct

SEQ ID NO: 29 - DNA, 110 bp, synthetic construct
  source 1..110: mol_type = other DNA
gtcacctttataccatttaacaaataaaaaaacgcaatcttggaaagcttagtacttccctgtagctcagatggtgaagaatctgcctgcgatgcaggagacctggggtt

SEQ ID NO: 30 - DNA, 110 bp, synthetic construct
  source 1..110: mol_type = other DNA
ccatttaacaaataaaaaaacgcaatcttggaaagcttagtacttccctgtagctcagatggtgaagaatctgcctgcgatgcaggagacctggggttgatccctgggtg

SEQ ID NO: 31 - DNA, 110 bp, synthetic construct
  source 1..110: mol_type = other DNA
tggtgtttagcgttcattatttgttgtgtgacatttctcttatattgataaatgaaatgtatgccctgtataaattagctttgggttttagatcctaaaatgcaacctaa

SEQ ID NO: 32 - DNA, 110 bp, synthetic construct
  source 1..110: mol_type = other DNA
gatgctttgcagtatcttctcattcaattttcatgaggacaccaaatctacaggctaaatacctaacttctgctaggtgctttgcagtatctcattcagttttcatgagg

SEQ ID NO: 33 - DNA, 110 bp, synthetic construct
  source 1..110: mol_type = other DNA
ttcaattttcatgaggacaccaaatctacaggctaaatacctaacttctgctaggtgctttgcagtatctcattcagttttcatgaggacaccgtgaagtctgttctgat

SEQ ID NO: 34 - DNA, 110 bp, synthetic construct
  source 1..110: mol_type = other DNA
aatctacaggctaaatacctaacttctgctaggtgctttgcagtatctcattcagttttcatgaggacaccgtgaagtctgttctgatcatgtctctcttattaatgagg

SEQ ID NO: 35 - DNA, 110 bp, synthetic construct
  source 1..110: mol_type = other DNA
cttctgctaggtgctttgcagtatctcattcagttttcatgaggacaccgtgaagtctgttctgatcatgtctctcttattaatgaggaaaatgaactttgaatggacct

SEQ ID NO: 36 - DNA, 110 bp, synthetic construct
  source 1..110: mol_type = other DNA
atctcattcagttttcatgaggacaccgtgaagtctgttctgatcatgtctctcttattaatgaggaaaatgaactttgaatggaccttgagcaattcacttccccgttc

SEQ ID NO: 37 - DNA, 110 bp, synthetic construct
  source 1..110: mol_type = other DNA
caactttaccttgactgtagagagccaagcattggagagggggaaaaaaagagcgctggattttccattctcttacccccactcctgggcctctactccaccaactctgg

SEQ ID NO: 38 - DNA, 110 bp, synthetic construct
  source 1..110: mol_type = other DNA
agccaagcattggagagggggaaaaaaagagcgctggattttccattctcttacccccactcctgggcctctactccaccaactctggttaccacggttgcggagtttct

SEQ ID NO: 39 - DNA, 110 bp, synthetic construct
  source 1..110: mol_type = other DNA
aaaaaagagcgctggattttccattctcttacccccactcctgggcctctactccaccaactctggttaccacggttgcggagtttctcaccactgaggtcacttggcat

SEQ ID NO: 40 - DNA, 110 bp, synthetic construct
  source 1..110: mol_type = other DNA
attctcttacccccactcctgggcctctactccaccaactctggttaccacggttgcggagtttctcaccactgaggtcacttggcatttctggggttgtgacacccaca

SEQ ID NO: 41 - DNA, 110 bp, synthetic construct
  source 1..110: mol_type = other DNA
gcctctactccaccaactctggttaccacggttgcggagtttctcaccactgaggtcacttggcatttctggggttgtgacacccacaggagccagaaacaataggcata

SEQ ID NO: 42 - DNA, 110 bp, synthetic construct
  source 1..110: mol_type = other DNA
tgggatcaggtagcaatgagatcgcttcggttaagacttggggaccccagcaactcttggttacagtatcaagcaggttgaagacatcagggatgtttattgtccctgag

SEQ ID NO: 43 - DNA, 110 bp, synthetic construct
  source 1..110: mol_type = other DNA
cgcttcggttaagacttggggaccccagcaactcttggttacagtatcaagcaggttgaagacatcagggatgtttattgtccctgagggagaaaaaaaatctatggagt

SEQ ID NO: 44 - DNA, 110 bp, synthetic construct
  source 1..110: mol_type = other DNA
ccccagcaactcttggttacagtatcaagcaggttgaagacatcagggatgtttattgtccctgagggagaaaaaaaatctatggagtttccaacgtcgttgttgttcag

SEQ ID NO: 45 - DNA, 110 bp, synthetic construct
  source 1..110: mol_type = other DNA
tatcaagcaggttgaagacatcagggatgtttattgtccctgagggagaaaaaaaatctatggagtttccaacgtcgttgttgttcagttgctcagtcatgtccagtgct

SEQ ID NO: 46 - DNA, 110 bp, synthetic construct
  source 1..110: mol_type = other DNA
tgaagacatcagggatgtttattgtccctgagggagaaaaaaaatctatggagtttccaacgtcgttgttgttcagttgctcagtcatgtccagtgctttgcaaccccat

SEQ ID NO: 47 - DNA, 110 bp, synthetic construct
  source 1..110: mol_type = other DNA
caaaagcaaaacacaaaacaggaagagtaaaggagaccagcggcaggaacagcacagggaaaaggcaagcagcaagtcctctgagagctgagaacaagagataaacaaac

SEQ ID NO: 48 - DNA, 110 bp, synthetic construct
  source 1..110: mol_type = other DNA
aagagtaaaggagaccagcggcaggaacagcacagggaaaaggcaagcagcaagtcctctgagagctgagaacaagagataaacaaacagaccagttctgtcctgtggaa

SEQ ID NO: 49 - DNA, 110 bp, synthetic construct
  source 1..110: mol_type = other DNA
aggaacagcacagggaaaaggcaagcagcaagtcctctgagagctgagaacaagagataaacaaacagaccagttctgtcctgtggaaagagagacaaatagcttcctgg

SEQ ID NO: 50 - DNA, 110 bp, synthetic construct
  source 1..110: mol_type = other DNA
aagcagcaagtcctctgagagctgagaacaagagataaacaaacagaccagttctgtcctgtggaaagagagacaaatagcttcctggttctcaaaactgtagcaacgga

SEQ ID NO: 51 - DNA, 110 bp, synthetic construct
  source 1..110: mol_type = other DNA
tgagaacaagagataaacaaacagaccagttctgtcctgtggaaagagagacaaatagcttcctggttctcaaaactgtagcaacggaaacatttacttatttatgcgtt

SEQ ID NO: 52 - DNA, 110 bp, synthetic construct
  source 1..110: mol_type = other DNA
tgagcttttggcgcctcctgccactgctcacacagcaccagccttctgggtggagggtgaacctggcctggcttgtctgacctggaagctccttgaggtcaggagcctgt

SEQ ID NO: 53 - DNA, 110 bp, synthetic construct
  source 1..110: mol_type = other DNA
actgctcacacagcaccagccttctgggtggagggtgaacctggcctggcttgtctgacctggaagctccttgaggtcaggagcctgtcttcttcagcccattatgccca

SEQ ID NO: 54 - DNA, 110 bp, synthetic construct
  source 1..110: mol_type = other DNA
tctgggtggagggtgaacctggcctggcttgtctgacctggaagctccttgaggtcaggagcctgtcttcttcagcccattatgcccaggtctagctgggtgcctcctgc

SEQ ID NO: 55 - DNA, 110 bp, synthetic construct
  source 1..110: mol_type = other DNA
cctggcttgtctgacctggaagctccttgaggtcaggagcctgtcttcttcagcccattatgcccaggtctagctgggtgcctcctgcatcttggatgccgggtgctcag

SEQ ID NO: 56 - DNA, 110 bp, synthetic construct
  source 1..110: mol_type = other DNA
ctccttgaggtcaggagcctgtcttcttcagcccattatgcccaggtctagctgggtgcctcctgcatcttggatgccgggtgctcaggaccacgtgaatgagaatgtcc

SEQ ID NO: 57 - DNA, 110 bp, synthetic construct
  source 1..110: mol_type = other DNA
gattaaaatctcaccactactgatcagagagctgtaggcaactcgctgcacatggcattgagctgtgaggcacaagcttctgaataatggaagctcaattgtctcccaag

SEQ ID NO: 58 - DNA, 110 bp, synthetic construct
  source 1..110: mol_type = other DNA
atcagagagctgtaggcaactcgctgcacatggcattgagctgtgaggcacaagcttctgaataatggaagctcaattgtctcccaagccaatttagaccaagacttagc

SEQ ID NO: 59 - DNA, 110 bp, synthetic construct
  source 1..110: mol_type = other DNA
gctgcacatggcattgagctgtgaggcacaagcttctgaataatggaagctcaattgtctcccaagccaatttagaccaagacttagctgaaaggtaagggcagtctctt

SEQ ID NO: 60 - DNA, 110 bp, synthetic construct
  source 1..110: mol_type = other DNA
gaggcacaagcttctgaataatggaagctcaattgtctcccaagccaatttagaccaagacttagctgaaaggtaagggcagtctcttcaggacgctcttactgagagac

SEQ ID NO: 61 - DNA, 110 bp, synthetic construct
  source 1..110: mol_type = other DNA
ggaagctcaattgtctcccaagccaatttagaccaagacttagctgaaaggtaagggcagtctcttcaggacgctcttactgagagaccctcccttaatgagggctcctg

SEQ ID NO: 62 - DNA, 110 bp, synthetic construct
  source 1..110: mol_type = other DNA
atggtcacattcacacaaatagtagctcattcttagaatggaaatgaagaatgcaaaaagtatgttttgatttttttgataactgaccttctctatacgtttgaattcac

SEQ ID NO: 63 - DNA, 110 bp, synthetic construct
  source 1..110: mol_type = other DNA
tagctcattcttagaatggaaatgaagaatgcaaaaagtatgttttgatttttttgataactgaccttctctatacgtttgaattcacacaactgagttcagagtttgtg

SEQ ID NO: 64 - DNA, 110 bp, synthetic construct
  source 1..110: mol_type = other DNA
tgaagaatgcaaaaagtatgttttgatttttttgataactgaccttctctatacgtttgaattcacacaactgagttcagagtttgtgtatgtgttacagctgcatattg

SEQ ID NO: 65 - DNA, 110 bp, synthetic construct
  source 1..110: mol_type = other DNA
ttgatttttttgataactgaccttctctatacgtttgaattcacacaactgagttcagagtttgtgtatgtgttacagctgcatattgtaagtaggggtagctcttattt

SEQ ID NO: 66 - DNA, 110 bp, synthetic construct
  source 1..110: mol_type = other DNA
ttctctatacgtttgaattcacacaactgagttcagagtttgtgtatgtgttacagctgcatattgtaagtaggggtagctcttatttggtcgaacatagattgattttc

SEQ ID NO: 67 - DNA, 110 bp, synthetic construct
  source 1..110: mol_type = other DNA
ccttcctaactgttgtattcaaaatgatttatggtttcaacttttagatattcctaagtctgtcctaaagttcaaattaaatagacccttttccaaaacttcttacactg

SEQ ID NO: 68 - DNA, 110 bp, synthetic construct
  source 1..110: mol_type = other DNA
aaagttcaaattaaatagacccttttccaaaacttcttacactgttttttccagatatattctttttgcctgtctatttttctaaatagcagagccttccttttcaccca

SEQ ID NO: 69 - DNA, 110 bp, synthetic construct
  source 1..110: mol_type = other DNA
ttttccaaaacttcttacactgttttttccagatatattctttttgcctgtctatttttctaaatagcagagccttccttttcacccaattcttcttccacttcacttat

SEQ ID NO: 70 - DNA, 110 bp, synthetic construct
  source 1..110: mol_type = other DNA
gctgagcacatctctccttggctgcccctgacctcatctccccagaggcagtaggaattctccccaaagaattcttccccacctcactgttgcgtacagggtcctctgca

SEQ ID NO: 71 - DNA, 110 bp, synthetic construct
  source 1..110: mol_type = other DNA
tgcccctgacctcatctccccagaggcagtaggaattctccccaaagaattcttccccacctcactgttgcgtacagggtcctctgcaatctcaggaaactcccgaggag

SEQ ID NO: 72 - DNA, 110 bp, synthetic construct
  source 1..110: mol_type = other DNA
gaggcagtaggaattctccccaaagaattcttccccacctcactgttgcgtacagggtcctctgcaatctcaggaaactcccgaggagttgacagggttcttctgctccc

SEQ ID NO: 73 - DNA, 110 bp, synthetic construct
  source 1..110: mol_type = other DNA
aagaattcttccccacctcactgttgcgtacagggtcctctgcaatctcaggaaactcccgaggagttgacagggttcttctgctccccgtgggcctgtgggtcacagta

SEQ ID NO: 74 - DNA, 110 bp, synthetic construct
  source 1..110: mol_type = other DNA
gttgcgtacagggtcctctgcaatctcaggaaactcccgaggagttgacagggttcttctgctccccgtgggcctgtgggtcacagtatgaccttttgcttatttcagtg

SEQ ID NO: 75 - DNA, 110 bp, synthetic construct
  source 1..110: mol_type = other DNA
actatgaagatggaattaaataatgcatggcaagctctgcttgccagaggttgacacttagtaagggtttaacagctgcaaattgcacattacctttgcggagagcgagg

SEQ ID NO: 76 - DNA, 110 bp, synthetic construct
  source 1..110: mol_type = other DNA
atgcatggcaagctctgcttgccagaggttgacacttagtaagggtttaacagctgcaaattgcacattacctttgcggagagcgaggcttgaggggaggaggtacatga

SEQ ID NO: 77 - DNA, 110 bp, synthetic construct
  source 1..110: mol_type = other DNA
cagaggttgacacttagtaagggtttaacagctgcaaattgcacattacctttgcggagagcgaggcttgaggggaggaggtacatgaggctggttttgaataccccacg

SEQ ID NO: 78 - DNA, 110 bp, synthetic construct
  source 1..110: mol_type = other DNA
gtttaacagctgcaaattgcacattacctttgcggagagcgaggcttgaggggaggaggtacatgaggctggttttgaataccccacggggtcagctctagcacttggct

SEQ ID NO: 79 - DNA, 110 bp, synthetic construct
  source 1..110: mol_type = other DNA
attacctttgcggagagcgaggcttgaggggaggaggtacatgaggctggttttgaataccccacggggtcagctctagcacttggctttgacaaacactgaagctgtgg

SEQ ID NO: 80 - DNA, 110 bp, synthetic construct
  source 1..110: mol_type = other DNA
aaccattggactgccagggaagcccctaccacttagttttaaatttgaattgcagttggattttcctagtgggttctttccctagttataagagtcagatgctcttaacc

SEQ ID NO: 81 - DNA, 110 bp, synthetic construct
  source 1..110: mol_type = other DNA
cccctaccacttagttttaaatttgaattgcagttggattttcctagtgggttctttccctagttataagagtcagatgctcttaaccaaaggaagtcttttgcaaggta

SEQ ID NO: 82 - DNA, 110 bp, synthetic construct
  source 1..110: mol_type = other DNA
ttgaattgcagttggattttcctagtgggttctttccctagttataagagtcagatgctcttaaccaaaggaagtcttttgcaaggtatgttttttaaacaaaacccagg

SEQ ID NO: 83 - DNA, 110 bp, synthetic construct
  source 1..110: mol_type = other DNA
tagtgggttctttccctagttataagagtcagatgctcttaaccaaaggaagtcttttgcaaggtatgttttttaaacaaaacccagggtgtggattcagacgatttgca

SEQ ID NO: 84 - DNA, 110 bp, synthetic construct
  source 1..110: mol_type = other DNA
taagagtcagatgctcttaaccaaaggaagtcttttgcaaggtatgttttttaaacaaaacccagggtgtggattcagacgatttgcatgtaagacctgtcagctgggta

SEQ ID NO: 85 - DNA, 110 bp, synthetic construct
  source 1..110: mol_type = other DNA
cgtgatctaccttctcacccacaaacaagtggagttgggcaaacaccatctttgctaaaaatgcacacaattccaagctcctcagtctctgaagtaggcgctgaggactt

SEQ ID NO: 86 - DNA, 110 bp, synthetic construct
  source 1..110: mol_type = other DNA
aaacaagtggagttgggcaaacaccatctttgctaaaaatgcacacaattccaagctcctcagtctctgaagtaggcgctgaggacttcagagctgggacagaataaccg

SEQ ID NO: 87 - DNA, 110 bp, synthetic construct
  source 1..110: mol_type = other DNA
accatctttgctaaaaatgcacacaattccaagctcctcagtctctgaagtaggcgctgaggacttcagagctgggacagaataaccgccagaaaaatgcagattgaatt

SEQ ID NO: 88 - DNA, 110 bp, synthetic construct
  source 1..110: mol_type = other DNA
acaattccaagctcctcagtctctgaagtaggcgctgaggacttcagagctgggacagaataaccgccagaaaaatgcagattgaattgtgacacccatcccaccctacc

SEQ ID NO: 89 - DNA, 110 bp, synthetic construct
  source 1..110: mol_type = other DNA
ctgaagtaggcgctgaggacttcagagctgggacagaataaccgccagaaaaatgcagattgaattgtgacacccatcccaccctacccccaccactcacaggctaggaa

SEQ ID NO: 90 - DNA, 110 bp, synthetic construct
  source 1..110: mol_type = other DNA
gtagggcggggccttgcgctcagctcctaggatcctcgaagctggcctggtatttagcctgtggtccccactgacaaggaaatggggacgtgacccagtgcattggaatg

SEQ ID NO: 91 - DNA, 110 bp, synthetic construct
  source 1..110: mol_type = other DNA
gctcctaggatcctcgaagctggcctggtatttagcctgtggtccccactgacaaggaaatggggacgtgacccagtgcattggaatgacacggtcctgtgtcaacctca

SEQ ID NO: 92 - DNA, 110 bp, synthetic construct
  source 1..110: mol_type = other DNA
gcctggtatttagcctgtggtccccactgacaaggaaatggggacgtgacccagtgcattggaatgacacggtcctgtgtcaacctcagtcccctcccggggaaacctca

SEQ ID NO: 93 - DNA, 110 bp, synthetic construct
  source 1..110: mol_type = other DNA
cccactgacaaggaaatggggacgtgacccagtgcattggaatgacacggtcctgtgtcaacctcagtcccctcccggggaaacctcaaccacctcccaccccaccaaca

SEQ ID NO: 94 - DNA, 110 bp, synthetic construct
  source 1..110: mol_type = other DNA
cgtgacccagtgcattggaatgacacggtcctgtgtcaacctcagtcccctcccggggaaacctcaaccacctcccaccccaccaacaaccctgccatctcttggggagc

SEQ ID NO: 95 - DNA, 110 bp, synthetic construct
  source 1..110: mol_type = other DNA
gcgaacataactgaggtttccgctgagattccttgccaaggcctccctagaatatttgcgcatcagtctccgggggcttccgctgtctggtcagctctcgatgagatcct

SEQ ID NO: 96 - DNA, 110 bp, synthetic construct
  source 1..110: mol_type = other DNA
ctgagattccttgccaaggcctccctagaatatttgcgcatcagtctccgggggcttccgctgtctggtcagctctcgatgagatcctcctacctgaggactccaggcct

SEQ ID NO: 97 - DNA, 110 bp, synthetic construct
  source 1..110: mol_type = other DNA
ccctagaatatttgcgcatcagtctccgggggcttccgctgtctggtcagctctcgatgagatcctcctacctgaggactccaggcctctcctgtggctgtgtcgtttgc

SEQ ID NO: 98 - DNA, 110 bp, synthetic construct
  source 1..110: mol_type = other DNA
tctccgggggcttccgctgtctggtcagctctcgatgagatcctcctacctgaggactccaggcctctcctgtggctgtgtcgtttgctctctgccactgtgaccgaggt

SEQ ID NO: 99 - DNA, 110 bp, synthetic construct
  source 1..110: mol_type = other DNA
ggtcagctctcgatgagatcctcctacctgaggactccaggcctctcctgtggctgtgtcgtttgctctctgccactgtgaccgaggtgtgtaactcggccccttctcct

SEQ ID NO: 100 - DNA, 110 bp, synthetic construct
  source 1..110: mol_type = other DNA
gggaggggaaggcatgctgtgtgagaggaatagtgtaaggaactgagtctgaagaccacaaacatggatagagtggaggatttattctttaccttctctacagtaaggca

SEQ ID NO: 101 - DNA, 110 bp, synthetic construct
  source 1..110: mol_type = other DNA
gagaggaatagtgtaaggaactgagtctgaagaccacaaacatggatagagtggaggatttattctttaccttctctacagtaaggcaatggtgaatgaactctgcctgt

SEQ ID NO: 102 - DNA, 110 bp, synthetic construct
  source 1..110: mol_type = other DNA
gagtctgaagaccacaaacatggatagagtggaggatttattctttaccttctctacagtaaggcaatggtgaatgaactctgcctgttaatgttgcacattgaaacact

SEQ ID NO: 103 - DNA, 110 bp, synthetic construct
  source 1..110: mol_type = other DNA
gatagagtggaggatttattctttaccttctctacagtaaggcaatggtgaatgaactctgcctgttaatgttgcacattgaaacactaactaaaattcgggctgcggct

SEQ ID NO: 104 - DNA, 110 bp, synthetic construct
  source 1..110: mol_type = other DNA
ttaccttctctacagtaaggcaatggtgaatgaactctgcctgttaatgttgcacattgaaacactaactaaaattcgggctgcggctgtgctactcatagtgccatatg

SEQ ID NO: 105 - DNA, 110 bp, synthetic construct
  source 1..110: mol_type = other DNA
agagagcagaagcgagcagctctgcgctttataacccagctctgcactttataacccagcttttcattaaaggagctattggcaaccctggaaagagtgggggaagcaga

SEQ ID NO: 106 - DNA, 110 bp, synthetic construct
  source 1..110: mol_type = other DNA
tgcgctttataacccagctctgcactttataacccagcttttcattaaaggagctattggcaaccctggaaagagtgggggaagcagaggggaaggagagaagtggggaa

SEQ ID NO: 107 - DNA, 110 bp, synthetic construct
  source 1..110: mol_type = other DNA
cactttataacccagcttttcattaaaggagctattggcaaccctggaaagagtgggggaagcagaggggaaggagagaagtggggaagagaatttctcgttcaaataat

SEQ ID NO: 108 - DNA, 110 bp, synthetic construct
  source 1..110: mol_type = other DNA
ttaaaggagctattggcaaccctggaaagagtgggggaagcagaggggaaggagagaagtggggaagagaatttctcgttcaaataatcccatggttttcagcaaggtca

SEQ ID NO: 109 - DNA, 110 bp, synthetic construct
  source 1..110: mol_type = other DNA
tggaaagagtgggggaagcagaggggaaggagagaagtggggaagagaatttctcgttcaaataatcccatggttttcagcaaggtcaaaagtctgagagttctgtggtg

SEQ ID NO: 110 - DNA, 110 bp, synthetic construct
  source 1..110: mol_type = other DNA
ggtttgaactggagtatacaggtgaatcacgatcttgtggggcttttttccttttcagcccaatgcaacttgattttcttggtgactggaagtttctttcctaaatctgt

SEQ ID NO: 111 - DNA, 110 bp, synthetic construct
  source 1..110: mol_type = other DNA
agtatacaggtgaatcacgatcttgtggggcttttttccttttcagcccaatgcaacttgattttcttggtgactggaagtttctttcctaaatctgtgaagctgtattt

SEQ ID NO: 112 - DNA, 110 bp, synthetic construct
  source 1..110: mol_type = other DNA
ttgtggggcttttttccttttcagcccaatgcaacttgattttcttggtgactggaagtttctttcctaaatctgtgaagctgtatttctttgggaaggagcttcacctg

SEQ ID NO: 113 - DNA, 110 bp, synthetic construct
  source 1..110: mol_type = other DNA
agcccaatgcaacttgattttcttggtgactggaagtttctttcctaaatctgtgaagctgtatttctttgggaaggagcttcacctgtgtctatttttaggttacacct

SEQ ID NO: 114 - DNA, 110 bp, synthetic construct
  source 1..110: mol_type = other DNA
tcctaaatctgtgaagctgtatttctttgggaaggagcttcacctgtgtctatttttaggttacacctagaggtgatatcacatgatgcgtgtgtctcccttacttcact

SEQ ID NO: 115 - DNA, 110 bp, synthetic construct
  source 1..110: mol_type = other DNA
ttgttagtgtcatagaattaaaggctgctgggaggaagatcaacagttgaaaaccctaaaaaaaagtgatagctgatgtttctcatttctttcttcaacgtaagtatatg

SEQ ID NO: 116 - DNA, 110 bp, synthetic construct
  source 1..110: mol_type = other DNA
ggctgctgggaggaagatcaacagttgaaaaccctaaaaaaaagtgatagctgatgtttctcatttctttcttcaacgtaagtatatgaattttaaaaaacttttaaatt

SEQ ID NO: 117 - DNA, 110 bp, synthetic construct
  source 1..110: mol_type = other DNA
taagcctgcgtggaacgcagagcctgcgcaatagcaccggtgaagttcccgatgccctgaactctgtttctctcagtgccaggcctggcagggttgtgcggctgtgtttg

SEQ ID NO: 118 - DNA, 110 bp, synthetic construct
  source 1..110: mol_type = other DNA
cctgcgcaatagcaccggtgaagttcccgatgccctgaactctgtttctctcagtgccaggcctggcagggttgtgcggctgtgtttggacggcagacgcccactgtggt

SEQ ID NO: 119 - DNA, 110 bp, synthetic construct
  source 1..110: mol_type = other DNA
gttcccgatgccctgaactctgtttctctcagtgccaggcctggcagggttgtgcggctgtgtttggacggcagacgcccactgtggtgctgcctcctcgcgggttctgg

SEQ ID NO: 120 - DNA, 110 bp, synthetic construct
  source 1..110: mol_type = other DNA
tttctctcagtgccaggcctggcagggttgtgcggctgtgtttggacggcagacgcccactgtggtgctgcctcctcgcgggttctggaagggagccagttcttggtttt

SEQ ID NO: 121 - DNA, 110 bp, synthetic construct
  source 1..110: mol_type = other DNA
cagggttgtgcggctgtgtttggacggcagacgcccactgtggtgctgcctcctcgcgggttctggaagggagccagttcttggttttagatgaaagggttaaatgccta

SEQ ID NO: 122 - DNA, 110 bp, synthetic construct
  source 1..110: mol_type = other DNA
ctatgacaaggaccatgctttgccttgtccctatgccttcacacctgctgttccatcaaactataatgctcctcccctgcttttcatctgagtcaatcccctttcatttg

SEQ ID NO: 123 - DNA, 110 bp, synthetic construct
  source 1..110: mol_type = other DNA
ccttgtccctatgccttcacacctgctgttccatcaaactataatgctcctcccctgcttttcatctgagtcaatcccctttcatttgagatgcaaaccaagtgtcaaat

SEQ ID NO: 124 - DNA, 110 bp, synthetic construct
  source 1..110: mol_type = other DNA
ctgctgttccatcaaactataatgctcctcccctgcttttcatctgagtcaatcccctttcatttgagatgcaaaccaagtgtcaaatatcatctcattccgtaagctct

SEQ ID NO: 125 - DNA, 110 bp, synthetic construct
  source 1..110: mol_type = other DNA
tgctcctcccctgcttttcatctgagtcaatcccctttcatttgagatgcaaaccaagtgtcaaatatcatctcattccgtaagctctcatataccctaactcactccag

SEQ ID NO: 126 - DNA, 110 bp, synthetic construct
  source 1..110: mol_type = other DNA
tgagtcaatcccctttcatttgagatgcaaaccaagtgtcaaatatcatctcattccgtaagctctcatataccctaactcactccagtaatcttgcctgggaaatccca

SEQ ID NO: 127 - DNA, 110 bp, synthetic construct
  source 1..110: mol_type = other DNA
cttcagtcaaggcagacggctggaaaacactgatcaaaggggaagcttggttagtttacactctccagctcgatttggatagaggcttcaggatgcttgcttctgacttg

SEQ ID NO: 128 - DNA, 110 bp, synthetic construct
  source 1..110: mol_type = other DNA
gaaaacactgatcaaaggggaagcttggttagtttacactctccagctcgatttggatagaggcttcaggatgcttgcttctgacttgatctgtaatcctagggaagctt

SEQ ID NO: 129 - DNA, 110 bp, synthetic construct
  source 1..110: mol_type = other DNA
gcttggttagtttacactctccagctcgatttggatagaggcttcaggatgcttgcttctgacttgatctgtaatcctagggaagcttcatgaaacacttgatagcctta

SEQ ID NO: 130 - DNA, 110 bp, synthetic construct
  source 1..110: mol_type = other DNA
agctcgatttggatagaggcttcaggatgcttgcttctgacttgatctgtaatcctagggaagcttcatgaaacacttgatagccttaaaccattcaagggctggttaaa

SEQ ID NO: 131 - DNA, 110 bp, synthetic construct
  source 1..110: mol_type = other DNA
caggatgcttgcttctgacttgatctgtaatcctagggaagcttcatgaaacacttgatagccttaaaccattcaagggctggttaaaaaaaataaatacagagttatga

SEQ ID NO: 132 - DNA, 110 bp, synthetic construct
  source 1..110: mol_type = other DNA
ggcactccctgggccagcttccccgctctgagcagggcagggtggagggtgagccagccgggaaggggttgccagtctgcagagactgagagccacgcgtgctcctgggc

SEQ ID NO: 133 - DNA, 110 bp, synthetic construct
  source 1..110: mol_type = other DNA
ccgctctgagcagggcagggtggagggtgagccagccgggaaggggttgccagtctgcagagactgagagccacgcgtgctcctgggcaggtgagcttcattcccatcct

SEQ ID NO: 134 - DNA, 110 bp, synthetic construct
  source 1..110: mol_type = other DNA
gagggtgagccagccgggaaggggttgccagtctgcagagactgagagccacgcgtgctcctgggcaggtgagcttcattcccatcctgagggctgggccggggagctgg

SEQ ID NO: 135 - DNA, 110 bp, synthetic construct
  source 1..110: mol_type = other DNA
ggttgccagtctgcagagactgagagccacgcgtgctcctgggcaggtgagcttcattcccatcctgagggctgggccggggagctgggcctgggagctggccctgggtt

SEQ ID NO: 136 - DNA, 110 bp, synthetic construct
  source 1..110: mol_type = other DNA
gcagagactgagagccacgcgtgctcctgggcaggtgagcttcattcccatcctgagggctgggccggggagctgggcctgggagctggccctgggtttcccctgcctgg

SEQ ID NO: 137 - DNA, 110 bp, synthetic construct
  source 1..110: mol_type = other DNA
ctgagaaagggactggagatgagagggaaggaggggataggagggatctgacgtggtgtttctacttctgagcactttgcagataaactgtccttaagaaggagcttctc

SEQ ID NO: 138 - DNA, 110 bp, synthetic construct
  source 1..110: mol_type = other DNA
gagggaaggaggggataggagggatctgacgtggtgtttctacttctgagcactttgcagataaactgtccttaagaaggagcttctcagtggagagcaaaatgtactca

SEQ ID NO: 139 - DNA, 110 bp, synthetic construct
  source 1..110: mol_type = other DNA
gatctgacgtggtgtttctacttctgagcactttgcagataaactgtccttaagaaggagcttctcagtggagagcaaaatgtactcagacctctgaatgttgcagggca

SEQ ID NO: 140 - DNA, 110 bp, synthetic construct
  source 1..110: mol_type = other DNA
tctgagcactttgcagataaactgtccttaagaaggagcttctcagtggagagcaaaatgtactcagacctctgaatgttgcagggcaaatgttactcaaagtctaataa

SEQ ID NO: 141 - DNA, 110 bp, synthetic construct
  source 1..110: mol_type = other DNA
tgtccttaagaaggagcttctcagtggagagcaaaatgtactcagacctctgaatgttgcagggcaaatgttactcaaagtctaataatttctttcaaagatttggatct

SEQ ID NO: 142 - DNA, 110 bp, synthetic construct
  source 1..110: mol_type = other DNA
tcagagcagggggccctgccgcagtggggaaggggggtctgcaccagccaccgcgtctcacctggcctgctccacgtcgcagatgtaggcctgccccaccacctcctcgt

SEQ ID NO: 143 - DNA, 110 bp, synthetic construct
  source 1..110: mol_type = other DNA
gggggtctgcaccagccaccgcgtctcacctggcctgctccacgtcgcagatgtaggcctgccccaccacctcctcgtggtacgtggctgcctccctcaacctcaactcg

SEQ ID NO: 144 - DNA, 110 bp, synthetic construct
  source 1..110: mol_type = other DNA
gtctcacctggcctgctccacgtcgcagatgtaggcctgccccaccacctcctcgtggtacgtggctgcctccctcaacctcaactcggaacaagccagggcaagtctgg

SEQ ID NO: 145 - DNA, 110 bp, synthetic construct
  source 1..110: mol_type = other DNA
tcgcagatgtaggcctgccccaccacctcctcgtggtacgtggctgcctccctcaacctcaactcggaacaagccagggcaagtctggggatgagaggagcgtcaggcct

SEQ ID NO: 146 - DNA, 110 bp, synthetic construct
  source 1..110: mol_type = other DNA
ccacctcctcgtggtacgtggctgcctccctcaacctcaactcggaacaagccagggcaagtctggggatgagaggagcgtcaggcctgagtcaggctgccctgggctca

SEQ ID NO: 147 - DNA, 110 bp, synthetic construct
  source 1..110: mol_type = other DNA
tggggtgacaaagagctggacacgactgaagcgccttaccacgcacacatgctcatgggagaatcaggtagacagcgccttgaagacacttaggggagtggtcagttctg

SEQ ID NO: 148 - DNA, 110 bp, synthetic construct
  source 1..110: mol_type = other DNA
cgactgaagcgccttaccacgcacacatgctcatgggagaatcaggtagacagcgccttgaagacacttaggggagtggtcagttctgcctttgggatggtgatagacag

SEQ ID NO: 149 - DNA, 110 bp, synthetic construct
  source 1..110: mol_type = other DNA
acacatgctcatgggagaatcaggtagacagcgccttgaagacacttaggggagtggtcagttctgcctttgggatggtgatagacaggaaagtctcccctgaggaactg

SEQ ID NO: 150 - DNA, 110 bp, synthetic construct
  source 1..110: mol_type = other DNA
ggtagacagcgccttgaagacacttaggggagtggtcagttctgcctttgggatggtgatagacaggaaagtctcccctgaggaactgacatttgagtcttaaaaatgtg

SEQ ID NO: 151 - DNA, 110 bp, synthetic construct
  source 1..110: mol_type = other DNA
cttaggggagtggtcagttctgcctttgggatggtgatagacaggaaagtctcccctgaggaactgacatttgagtcttaaaaatgtgaatatgcagatcaatcaagcgg

SEQ ID NO: 152 - DNA, 110 bp, synthetic construct
  source 1..110: mol_type = other DNA
cagctgctgaagtgcacaccccgagaagccaccgctcagcagccaagacgggcctccactcggccaactggagaaagcccacacagaagcaacgaagacgcagcacagcc

SEQ ID NO: 153 - DNA, 110 bp, synthetic construct
  source 1..110: mol_type = other DNA
gagaagccaccgctcagcagccaagacgggcctccactcggccaactggagaaagcccacacagaagcaacgaagacgcagcacagccaaggacgcgccgttcctgattc

SEQ ID NO: 154 - DNA, 110 bp, synthetic construct
  source 1..110: mol_type = other DNA
aagacgggcctccactcggccaactggagaaagcccacacagaagcaacgaagacgcagcacagccaaggacgcgccgttcctgattcttttcctgacggtgcctgctac

SEQ ID NO: 155 - DNA, 110 bp, synthetic construct
  source 1..110: mol_type = other DNA
actggagaaagcccacacagaagcaacgaagacgcagcacagccaaggacgcgccgttcctgattcttttcctgacggtgcctgctactcttagcatgttattgcaaagt

SEQ ID NO: 156 - DNA, 110 bp, synthetic construct
  source 1..110: mol_type = other DNA
gcaacgaagacgcagcacagccaaggacgcgccgttcctgattcttttcctgacggtgcctgctactcttagcatgttattgcaaagtcattcacatccacctcttttaa

(19) United States
(12) Patent Application Publication
(10) Pub. No.: US 2025/0163520 A1
(43) Pub. Date: May 22, 2025

(54) COMBINATION OF SINGLE NUCLEOTIDE POLYMORPHISM (SNP) LOCISASSOCIATED WITH DIAMETER OF WOOL FIBER OF FINE WOOL SHEEP, AND USE THEREOF

(71) Applicant: LANZHOU INSTITUTE OF ANIMAL HUSBANDRY AND VETERINARY MEDICINE, CAAS, LANZHOU (CN)

(72) Inventors: TINGTING GUO, LANZHOU (CN); CHAO YUAN, LANZHOU (CN); JIANBIN LIU, LANZHOU (CN); YAOJING YUE, LANZHOU (CN); ZENGKUI LU, LANZHOU (CN); CHUNE NIU, LANZHOU (CN); JIANYE LI, LANZHOU (CN); XIAOPING SUN, LANZHOU (CN); BINGJIE MA, LANZHOU (CN); BOHUI YANG, LANZHOU (CN)

(73) Assignee: LANZHOU INSTITUTE OF ANIMAL HUSBANDRY AND VETERINARY MEDICINE, CAAS, LANZHOU (CN)

(21) Appl. No.: 18/963,961

(22) Filed: Nov. 29, 2024

Related U.S. Application Data
(63) Continuation of application No. PCT/CN2023/096722, filed on May 29, 2023.

(51) Int. Cl. C12Q 1/6888; C12Q 1/6827; G16B 20/20; G16B 30/10

(52) U.S. Cl. CPC C12Q 1/6888; C12Q 1/6827; G16B 20/20; G16B 30/10; C12Q 2600/124; C12Q 2600/156

(57) ABSTRACT

The present disclosure pertains to the biological field, specifically to a combination of 33 single nucleotide polymorphism (SNP) loci associated with the wool fiber diameter of fine wool sheep, determined using Ovis aries reference genome version 4.0. It also covers the use of reagents for detecting genotypes of these SNP loci in wool fiber diameter analysis or molecular marker-assisted selection of fine wool sheep. Additionally, it includes molecular probe combinations, gene chips, and kits based on the SNP loci. These tools facilitate early-stage wool fiber trait analysis, enabling individual selection, reducing generation intervals, accelerating breeding processes, and lowering costs. This disclosure provides crucial support for the identification, breed preservation, and molecular genetic breeding of fine wool sheep.

### CROSS REFERENCE TO RELATED APPLICATION

The present application claims priority to a PCT international patent application PCT/CN2023/096722, filed on May 29, 2023, entitled “COMBINATION OF SINGLE NUCLEOTIDE POLYMORPHISM (SNP) LOCIS ASSOCIATED WITH DIAMETER OF WOOL FIBER OF FINE WOOL SHEEP, AND USE THEREOF”, which is incorporated herein by reference in its entirety.

### TECHNICAL FIELD

The present disclosure belongs to the technical field of genetic breeding, and specifically relates to a combination of single nucleotide polymorphism (SNP) locis associated with a wool fiber diameter of fine wool sheep, and a use thereof.

### BACKGROUND

Ovis aries is a domestic animal with significant agricultural and biological significances. As one of the first domesticated animals, Ovis aries provides meat, milk, wool, and lambskin for humans, and plays a vital role in the global agricultural economy. Wool is a source of high-quality textile raw materials and plays a significant role in the national economy.

There are abundant wool sheep breed resources in China. Wool is a natural high-performance material with various properties such as anti-fouling performance, softness, sun protection, warmth, and air permeability. Therefore, wool occupies an important place in textile processing. A quality of wool is determined by a fiber diameter, a fiber length, a degree of crimp, a color, and a medulla percentage. The fiber diameter is often related to processability of wool and determines the end use of wool. The above wool traits are affected by a combination of genetic and non-genetic factors. The fiber diameter, as one of the important economic traits of fine wool sheep, usually determines 75% to 80% of a unit value of wool. The finer the diameter of the fiber, the greater the economic value of the wool.

The fiber diameter is a major decisive factor for a quality and value of wool. An average fiber diameter is one of the most important properties of raw wool that can be measured. Therefore, the average fiber diameter is an important decisive factor for a price of greasy wool. The fiber diameter is also one of the few raw wool parameters that remains basically unchanged during processing. A diameter of a given raw material limits a thickness (count) of a yarn that can be spun from the raw material. For a given yarn count, various physical properties such as bending stiffness and elongation of a yarn depend on a diameter of fibers constituting the yarn.

Molecular genetic markers are based on nucleotide sequence variations in individual genetic materials, and are direct responses to genetic polymorphisms at a DNA level. The molecular genetic markers have significant advantages, for example, DNA of different tissues at different stages of biological development can be used for genetic marker analysis; there are abundant genomic variations; and detection manners are simple, fast, and easy to be automatized. At present, molecular genetic markers widely used include restriction fragment length polymorphism (RFLP), random amplified polymorphic DNA (RAPD), amplified fragment length polymorphism (AFLP), and SNP. SNPs are an important basis for investigating genetic variations in human families and animal and plant strains, and thus are often widely used in population genetic research and disease-associated gene exploration. In recent years, SNPs often play an important role in animal genetic analysis and genetic breeding. Therefore, SNPs are often used in the genetic breeding for domestic animals to accelerate the innovation of the traditional breeding technology and establish innovative breeding theories and systems.

Currently, although there are a large number of techniques for SNP genotyping, there is a lack of studies related to a specific trait. In particular, there is a lack of research on SNP locis combinations associated with a wool fiber diameter of fine wool sheep in the current sheep SNP research.

### SUMMARY

In order to meet the needs of chip locus function detection and function research in a direction of production traits during the current breeding production in China, the present disclosure provides high-depth whole-genome resequencing data of four representative Chinese fine wool sheep breeds (Chinese Merino sheep, Alpine Merino sheep, Aohan fine wool sheep, and Qinghai fine wool sheep), and uses the Ovis aries reference genome version 4.0 sequence as a reference to acquire a combination of SNP locis associated with a wool fiber diameter of fine wool sheep that has accurate detection, easy use, and promising market prospects in combination with the existing studies related to sheep production traits. The combination of SNP locis can be used for genome selective breeding, protection, and improvement of sheep breed. The present disclosure specifically includes the following contents:

In a first aspect, the present disclosure provides a combination of 33 SNP locis associated with a wool fiber diameter of fine wool sheep, where the combination of 33 SNP locis is determined based on alignment with an Ovis aries reference genome versions 4.0 sequence, and the 33 SNP locis are located at the following positions, respectively: position 203825947 of chr 1, with a deoxynucleotide of C or A; position 226733906 of chr 1, with a deoxynucleotide of C or T; position 45470146 of chr 3, with a deoxynucleotide of A or C; position 68142771 of chr 4, with a deoxynucleotide of C or G; position 93335425 of chr 5, with a deoxynucleotide of C or T; position 93344882 of chr 5, with a deoxynucleotide of C or T; position 93387255 of chr 5, with a deoxynucleotide of C or T; position 93391985 of chr 5, with a deoxynucleotide of G or A; position 93392877 of chr 5, with a deoxynucleotide of C or T; position 93393426 of chr 5, with a deoxynucleotide of A or G; position 93507537 of chr 5, with a deoxynucleotide of A or C; position 25952072 of chr 6, with a deoxynucleotide of T or A; position 37126564 of chr 6, with a deoxynucleotide of T or C; position 51739659 of chr 10, with a deoxynucleotide of G or A; position 8917643 of chr 11, with a deoxynucleotide of C or A; position 25119445 of chr 12, with a deoxynucleotide of C or A; position 25120732 of chr 12, with a deoxynucleotide of C or G; position 25135944 of chr 12, with a deoxynucleotide of A or G; position 25149517 of chr 12, with a deoxynucleotide of C or A; position 25152554 of chr 12, with a deoxynucleotide of T or A; position 25154575 of chr 12, with a deoxynucleotide of C or T; position 25155325 of chr 12, with a deoxynucleotide of T or C; position 36292909 of chr 12, with a deoxynucleotide of G or A; position 78576808 of chr 12, with a deoxynucleotide of T or C; position 68745093 of chr 15, with a deoxynucleotide of C or T; position 25185724 of chr 20, with a deoxynucleotide of C or T; position 37863763 of chr 20, with a deoxynucleotide of C or T; position 44119346 of chr 20, with a deoxynucleotide of G or A; position 46988971 of chr 22, with a deoxynucleotide of G or A; position 47002481 of chr 22, with a deoxynucleotide of A or C; position 50203275 of chr 22, with a deoxynucleotide of G or A; position 13791395 of chr 25, with a deoxynucleotide of T or C; and position 40609491 of chr 26, with a deoxynucleotide of C or T.

| | | | | | | SEQ |
|---|---|---|---|---|---|---|
| Number | Mark | Chr | start | end | Probe Information | ID NO |
| SNP00001 | 1_ | 1 | 203825849 | 203825958 | CCATCTATTATTTTATTAATTTATCTC | SEQ |
| | 203825947 | | | | CTCCGAACTGCCTAGTAGTAGCAC | ID |
| | | | | | TGATACTGCAAATACACAGATATTT | NO. 1 |
| | | | | | CAAATGGGCATTTAAAAGGGCAGT | |
| | | | | | TTCCATGGGC | |
| | | | 203825871 | 203825980 | ATCTCCTCCGAACTGCCTAGTAGTA | SEQ |
| | | | | | GCACTGATACTGCAAATACACAGA | ID |
| | | | | | TATTTCAAATGGGCATTTAAAAGG | NO. 2 |
| | | | | | GCAGTTTCCATGGGCTCATTTTGA | |
| | | | | | GTTGCAACAGAGG | |
| | | | 203825893 | 203826002 | GTAGCACTGATACTGCAAATACAC | SEQ |
| | | | | | AGATATTTCAAATGGGCATTTAAAA | ID |
| | | | | | GGGCAGTTTCCATGGGCTCATTTT | NO. 3 |
| | | | | | GAGTTGCAACAGAGGTTTATCAGA | |
| | | | | | TATATCAAGGTTT | |
| | | | 203825915 | 203826024 | ACAGATATTTCAAATGGGCATTTAA | SEQ |
| | | | | | AAGGGCAGTTTCCATGGGCTCATT | ID |
| | | | | | TTGAGTTGCAACAGAGGTTTATCA | NO. 4 |
| | | | | | GATATATCAAGGTTTTACCCTTTCT | |
| | | | | | TTAAGGATAGAG | |
| | | | 203825937 | 203826046 | TAAAAGGGCAGTTTCCATGGGCTC | SEQ |
| | | | | | ATTTTGAGTTGCAACAGAGGTTTA | ID |
| | | | | | TCAGATATATCAAGGTTTTACCCTT | NO. 5 |
| | | | | | TCTTTAAGGATAGAGGCCAAATCT | |
| | | | | | AGTATTGTTTATA | |
| SNP | 1_ | 1 | 226733808 | 226733917 | TGAGAGCTGTTTAGTAATAATGCA | SEQ |
| 00002 | 226733906 | | | | GTTTTCTTGAGGTCTGTTTATTTTAT | ID |
| | | | | | AGTTTTTTAAAAAACCTTCAGTTC | NO. 6 |
| | | | | | TTTCGGCCAGCGTGTGTGTATTATC | |
| | | | | | TGTGCATTAAT | |
| | | | 226733830 | 226733939 | CAGTTTTCTTGAGGTCTGTTTATTT | SEQ |
| | | | | | TATAGTTTTTTAAAAAACCTTCAGT | ID |
| | | | | | TCTTTCGGCCAGCGTGTGTGTATTA | NO. 7 |
| | | | | | TCTGTGCATTAATGGTCCTCATCTG | |
| | | | | | ACTACTGCAT | |
| | | | 226733852 | 226733961 | TTTTATAGTTTTTTAAAAAACCTTC | SEQ |
| | | | | | AGTTCTTTCGGCCAGCGTGTGTGT | ID |
| | | | | | ATTATCTGTGCATTAATGGTCCTCA | NO. 8 |
| | | | | | TCTGACTACTGCATTGGTTCATGTT | |
| | | | | | TTTCTGCATGG | |
| | | | 226733874 | 226733983 | TTCAGTTCTTTCGGCCAGCGTGTG | SEQ |
| | | | | | TGTATTATCTGTGCATTAATGGTCC | ID |
| | | | | | TCATCTGACTACTGCATTGGTTCAT | NO. 9 |
| | | | | | GTTTTTCTGCATGGATTGACATAAA | |
| | | | | | ACCATTACTAA | |
| | | | 226733896 | 226734005 | TGTGTATTATCTGTGCATTAATGGT | SEQ |
| | | | | | CCTCATCTGACTACTGCATTGGTTC | ID |
| | | | | | ATGTTTTTCTGCATGGATTGACATA | NO. 10 |
| | | | | | AAACCATTACTAAAATTTGGCACC | |
| | | | | | TATGAGATATC | |
| SNP00003 | 3_ | 3 | 45470048 | 45470157 | CGTGAAAGGGCCATATTAATCATTA | SEQ |
| | 45470146 | | | | GGCAAGACAAACCACGGAAATTC | ID |
| | | | | | CTATTAAGCAACATCTCAGTTCTCC | NO. 11 |
| | | | | | CCAGAAAGTCCCCCTAAGGCTACT | |
| | | | | | TATTCACTCTTTA | |
| | | | 45470070 | 45470179 | TTAGGCAAGACAAACCACGGAAA | SEQ |
| | | | | | TTCCTATTAAGCAACATCTCAGTTC | ID |
| | | | | | TCCCCAGAAAGTCCCCCTAAGGCT | NO. 12 |
| | | | | | ACTTATTCACTCTTTAATCTTGACT | |
| | | | | | ACACATAGGCTCC | |
| | | | 45470092 | 45470201 | ATTCCTATTAAGCAACATCTCAGTT | SEQ |
| | | | | | CTCCCCAGAAAGTCCCCCTAAGGC | ID |
| | | | | | TACTTATTCACTCTTTAATCTTGAC | NO. 13 |
| | | | | | TACACATAGGCTCCAGTTTTCCCTG | |
| | | | | | GCTCTGGATCT | |
| | | | 45470114 | 45470223 | GTTCTCCCCAGAAAGTCCCCCTAA | SEQ |
| | | | | | GGCTACTTATTCACTCTTTAATCTT | ID |
| | | | | | GACTACACATAGGCTCCAGTTTTC | NO. 14 |
| | | | | | CCTGGCTCTGGATCTCACTGCATTG | |
| | | | | | TCCCTTGGTCCT | |
| | | | 45470136 | 45470245 | AAGGCTACTTATTCACTCTTTAATC | SEQ |
| | | | | | TTGACTACACATAGGCTCCAGTTTT | ID |
| | | | | | CCCTGGCTCTGGATCTCACTGCATT | NO. 15 |
| | | | | | GTCCCTTGGTCCTGCTCTCCCCCTT | |
| | | | | | ACCTCCTGCA | |
| SNP00004 | 4_ | 4 | 68142673 | 68142782 | AAAAAAGAAACACATGCGGACAT | SEQ |
| | 68142771 | | | | AGTCCACACACTCACAGACACAG | ID |
| | | | | | ATACCATGATAAATTCCAGACCACA | NO. 16 |
| | | | | | AGACAGACTGGGAAAGTCTCCCTC | |
| | | | | | TCTCCTTTTTTTTTT | |
| | | | 68142695 | 68142804 | TAGTCCACACACTCACAGACACAG | SEQ |
| | | | | | ATACCATGATAAATTCCAGACCACA | ID |
| | | | | | AGACAGACTGGGAAAGTCTCCCTC | NO. 17 |
| | | | | | TCTCCTTTTTTTTTTTTTAATGAATT | |
| | | | | | TTCTTGAGATT | |
| | | | 68142717 | 6842826 | AGATACCATGATAAATTCCAGACC | SEQ |
| | | | | | ACAAGACAGACTGGGAAAGTCTC | ID |
| | | | | | CCTCTCTCCTTTTTTTTTTTTTAATG | NO. 18 |
| | | | | | AATTTTCTTGAGATTAGCTTCAGCA | |
| | | | | | TTTTAGCCAGTA | |
| | | | 68142739 | 68142848 | CCACAAGACAGACTGGGAAAGTC | SEQ |
| | | | | | TCCCTCTCTCCTTTTTTTTTTTTTAA | ID |
| | | | | | TGAATTTTCTTGAGATTAGCTTCAG | NO. 19 |
| | | | | | CATTTTAGCCAGTATTTTAAGGACT | |
| | | | | | AACAGACTTAT | |
| | | | 68142761 | 68142870 | CTCCCTCTCTCCTTTTTTTTTTTTTA | SEQ |
| | | | | | ATGAATTTTCTTGAGATTAGCTTCA | ID |
| | | | | | GCATTTTAGCCAGTATTTTAAGGAC | NO. 20 |
| | | | | | TAACAGACTTATGAGTTACCATCAT | |
| | | | | | TGGCATCTA | |
| SNP00005 | 5_ | 5 | 93335327 | 93335436 | TTTTTTCTCTGTGAATAGCTTTTAA | SEQ |
| | 93335425 | | | | CTAAAAACTGTCAGTTACCCAGTG | ID |
| | | | | | TTTCTCAACAGAGTACTAATGGCAT | NO. 21 |
| | | | | | TTGGGTTGACAGTTCTTAATTGGG | |
| | | | | | CGGGCTCTCTTG | |
| | | | 93335349 | 93335458 | TAACTAAAAACTGTCAGTTACCCA | SEQ |
| | | | | | GTGTTTCTCAACAGAGTACTAATG | ID |
| | | | | | GCATTTGGGTTGACAGTTCTTAATT | NO. 22 |
| | | | | | GGGCGGGCTCTCTTGTTCCCACTA | |
| | | | | | AATGCCAGAGCGT | |
| | | | 93335371 | 93335480 | CAGTGTTTCTCAACAGAGTACTAA | SEQ |
| | | | | | TGGCATTTGGGTTGACAGTTCTTA | ID |
| | | | | | ATTGGGCGGGCTCTCTTGTTCCCA | NO. 23 |
| | | | | | CTAAATGCCAGAGCGTGACAAAA | |
| | | | | | ACGCTGTCATGCACA | |
| | | | 93335393 | 93335502 | AATGGCATTTGGGTTGACAGTTCT | SEQ |
| | | | | | TAATTGGGCGGGCTCTCTTGTTCCC | ID |
| | | | | | ACTAAATGCCAGAGCGTGACAAA | NO. 24 |
| | | | | | AACGCTGTCATGCACACATTTTAA | |
| | | | | | AATACTCCTTCATT | |
| | | | 93335415 | 93335524 | CTTAATTGGGCGGGCTCTCTTGTTC | SEQ |
| | | | | | CCACTAAATGCCAGAGCGTGACAA | ID |
| | | | | | AAACGCTGTCATGCACACATTTTA | NO. 25 |
| | | | | | AAATACTCCTTCATTGGATGCTGCC | |
| | | | | | CCCCTCCCCCGC | |
| SNP00006 | 5_ | 5 | 93344784 | 93344893 | TTATGATGTGCCAGGCCTGATGAA | SEQ |
| | 93344882 | | | | CACACCCTCATTAATTCCTCACTAC | ID |
| | | | | | CATCTTATGAGGTAGATGTCACCTT | NO. 26 |
| | | | | | TATACCATTTAACAAATAAAAAAAC | |
| | | | | | GCAATCTTGGA | |
| | | | 93344806 | 93344915 | AACACACCCTCATTAATTCCTCACT | SEQ |
| | | | | | ACCATCTTATGAGGTAGATGTCACC | ID |
| | | | | | TTTATACCATTTAACAAATAAAAAA | NO. 27 |
| | | | | | ACGCAATCTTGGAAAGCTTAGTAC | |
| | | | | | TTCCCTGTAGC | |
| | | | 93344828 | 93344937 | ACTACCATCTTATGAGGTAGATGTC | SEQ |
| | | | | | ACCTTTATACCATTTAACAAATAAA | ID |
| | | | | | AAAACGCAATCTTGGAAAGCTTAG | NO. 28 |
| | | | | | TACTTCCCTGTAGCTCAGATGGTG | |
| | | | | | AAGAATCTGCCT | |
| | | | 93344850 | 93344959 | GTCACCTTTATACCATTTAACAAAT | SEQ |
| | | | | | AAAAAAACGCAATCTTGGAAAGC | ID |
| | | | | | TTAGTACTTCCCTGTAGCTCAGATG | NO. 29 |
| | | | | | GTGAAGAATCTGCCTGCGATGCAG | |
| | | | | | GAGACCTGGGGTT | |
| | | | 93344862 | 93344971 | CCATTTAACAAATAAAAAAACGCA | SEQ |
| | | | | | ATCTTGGAAAGCTTAGTACTTCCCT | ID |
| | | | | | GTAGCTCAGATGGTGAAGAATCTG | NO. 30 |
| | | | | | CCTGCGATGCAGGAGACCTGGGGT | |
| | | | | | TGATCCCTGGGTG | |
| SNP00007 | 5_ | 5 | 93387245 | 93387354 | TGGTGTTTAGCGTTCATTATTTGTT | SEQ |
| | 93387255 | | | | GTGTGACATTTCTCTTATATTGATA | ID |
| | | | | | AATGAAATGTATGCCCTGTATAAAT | NO. 31 |
| | | | | | TAGCTTTGGGTTTTAGATCCTAAAA | |
| | | | | | TGCAACCTAA | |
| SNP00008 | 5_ | 5 | 93391887 | 93391996 | GATGCTTTGCAGTATCTTCTCATTC | SEQ |
| | 93391985 | | | | AATTTTCATGAGGACACCAAATCT | ID |
| | | | | | ACAGGCTAAATACCTAACTTCTGC | NO. 32 |
| | | | | | TAGGTGCTTTGCAGTATCTCATTCA | |
| | | | | | GTTTTCATGAGG | |
| | | | 93391909 | 93392018 | TTCAATTTTCATGAGGACACCAAA | SEQ |
| | | | | | TCTACAGGCTAAATACCTAACTTCT | ID |
| | | | | | GCTAGGTGCTTTGCAGTATCTCATT | NO. 33 |
| | | | | | CAGTTTTCATGAGGACACCGTGAA | |
| | | | | | GTCTGTTCTGAT | |
| | | | 93391931 | 93392040 | AATCTACAGGCTAAATACCTAACTT | SEQ |
| | | | | | CTGCTAGGTGCTTTGCAGTATCTCA | ID |
| | | | | | TTCAGTTTTCATGAGGACACCGTG | NO. 34 |
| | | | | | AAGTCTGTTCTGATCATGTCTCTCT | |
| | | | | | TATTAATGAGG | SEQ |
| | | | 93391953 | 93392062 | CTTCTGCTAGGTGCTTTGCAGTATC | ID |
| | | | | | TCATTCAGTTTTCATGAGGACACC | |
| | | | | | GTGAAGTCTGTTCTGATCATGTCTC | NO. 35 |
| | | | | | TCTTATTAATGAGGAAAATGAACTT | |
| | | | | | TGAATGGACCT | |
| | | | 93391975 | 93392084 | ATCTCATTCAGTTTTCATGAGGACA | SEQ |
| | | | | | CCGTGAAGTCTGTTCTGATCATGTC | ID |
| | | | | | TCTCTTATTAATGAGGAAAATGAAC | NO. 36 |
| | | | | | TTTGAATGGACCTTGAGCAATTCA | |
| | | | | | CTTCCCCGTTC | |
| SNP00009 | 5_ | 5 | 93392779 | 93392888 | CAACTTTACCTTGACTGTAGAGAG | SEQ |
| | 93392877 | | | | CCAAGCATTGGAGAGGGGGAAAA | ID |
| | | | | | AAAGAGCGCTGGATTTTCCATTCT | NO. 37 |
| | | | | | CTTACCCCCACTCCTGGGCCTCTAC | |
| | | | | | TCCACCAACTCTGG | |
| | | | 93392801 | 93392910 | AGCCAAGCATTGGAGAGGGGGAA | SEQ |
| | | | | | AAAAAGAGCGCTGGATTTTCCATT | ID |
| | | | | | CTCTTACCCCCACTCCTGGGCCTCT | NO. 38 |
| | | | | | ACTCCACCAACTCTGGTTACCACG | |
| | | | | | GTTGCGGAGTTTCT | |
| | | | 93392823 | 93392932 | AAAAAAGAGCGCTGGATTTTCCAT | SEQ |
| | | | | | TCTCTTACCCCCACTCCTGGGCCTC | ID |
| | | | | | TACTCCACCAACTCTGGTTACCAC | NO. 39 |
| | | | | | GGTTGCGGAGTTTCTCACCACTGA | |
| | | | | | GGTCACTTGGCAT | |
| | | | 93392845 | 93392954 | ATTCTCTTACCCCCACTCCTGGGCC | SEQ |
| | | | | | TCTACTCCACCAACTCTGGTTACC | ID |
| | | | | | ACGGTTGCGGAGTTTCTCACCACT | NO. 40 |
| | | | | | GAGGTCACTTGGCATTTCTGGGGT | |
| | | | | | TGTGACACCCACA | |
| | | | 93392867 | 93392976 | GCCTCTACTCCACCAACTCTGGTT | SEQ |
| | | | | | ACCACGGTTGCGGAGTTTCTCACC | ID |
| | | | | | ACTGAGGTCACTTGGCATTTCTGG | NO. 41 |
| | | | | | GGTTGTGACACCCACAGGAGCCA | |
| | | | | | GAAACAATAGGCATA | |
| SNP00010 | 5_ | 5 | 93393328 | 93393437 | TGGGATCAGGTAGCAATGAGATCG | SEQ |
| | 93393426 | | | | CTTCGGTTAAGACTTGGGGACCCC | ID |
| | | | | | AGCAACTCTTGGTTACAGTATCAA | NO. 42 |
| | | | | | GCAGGTTGAAGACATCAGGGATGT | |
| | | | | | TTATTGTCCCTGAG | |
| | | | 93393350 | 93393459 | CGCTTCGGTTAAGACTTGGGGACC | SEQ |
| | | | | | CCAGCAACTCTTGGTTACAGTATC | ID |
| | | | | | AAGCAGGTTGAAGACATCAGGGAT | NO. 43 |
| | | | | | GTTTATTGTCCCTGAGGGAGAAAA | |
| | | | | | AAAATCTATGGAGT | |
| | | | 93393372 | 93393481 | CCCCAGCAACTCTTGGTTACAGTA | SEQ |
| | | | | | TCAAGCAGGTTGAAGACATCAGG | ID |
| | | | | | GATGTTTATTGTCCCTGAGGGAGA | NO. 44 |
| | | | | | AAAAAAATCTATGGAGTTTCCAAC | |
| | | | | | GTCGTTGTTGTTCAG | |
| | | | 93393394 | 93393503 | TATCAAGCAGGTTGAAGACATCAG | SEQ |
| | | | | | GGATGTTTATTGTCCCTGAGGGAG | ID |
| | | | | | AAAAAAAATCTATGGAGTTTCCAA | NO. 45 |
| | | | | | CGTCGTTGTTGTTCAGTTGCTCAG | |
| | | | | | TCATGTCCAGTGCT | |
| | | | 93393406 | 93393515 | TGAAGACATCAGGGATGTTTATTG | SEQ |
| | | | | | TCCCTGAGGGAGAAAAAAAATCTA | ID |
| | | | | | TGGAGTTTCCAACGTCGTTGTTGT | NO. 46 |
| | | | | | TCAGTTGCTCAGTCATGTCCAGTG | |
| | | | | | CTTTGCAACCCCAT | |
| SNP | 5_ | 5 | 93507439 | 93507548 | CAAAAGCAAAACACAAAACAGGA | SEQ |
| 00011 | 93507537 | | | | AGAGTAAAGGAGACCAGCGGCAG | ID |
| | | | | | GAACAGCACAGGGAAAAGGCAAG | NO. 47 |
| | | | | | CAGCAAGTCCTCTGAGAGCTGAG | |
| | | | | | AACAAGAGATAAACAAAC | |
| | | | 93507461 | 93507570 | AAGAGTAAAGGAGACCAGCGGCA | SEQ |
| | | | | | GGAACAGCACAGGGAAAAGGCAA | ID |
| | | | | | GCAGCAAGTCCTCTGAGAGCTGA | NO. 48 |
| | | | | | GAACAAGAGATAAACAAACAGAC | |
| | | | | | CAGTTCTGTCCTGTGGAA | |
| | | | 93507483 | 93507592 | AGGAACAGCACAGGGAAAAGGCA | SEQ |
| | | | | | AGCAGCAAGTCCTCTGAGAGCTG | ID |
| | | | | | AGAACAAGAGATAAACAAACAGA | NO. 49 |
| | | | | | CCAGTTCTGTCCTGTGGAAAGAGA | |
| | | | | | GACAAATAGCTTCCTGG | |
| | | | 93507505 | 93507614 | AAGCAGCAAGTCCTCTGAGAGCT | SEQ |
| | | | | | GAGAACAAGAGATAAACAAACAG | ID |
| | | | | | ACCAGTTCTGTCCTGTGGAAAGAG | NO. 50 |
| | | | | | AGACAAATAGCTTCCTGGTTCTCA | |
| | | | | | AAACTGTAGCAACGGA | |
| | | | 93507527 | 93507636 | TGAGAACAAGAGATAAACAAACA | SEQ |
| | | | | | GACCAGTTCTGTCCTGTGGAAAGA | ID |
| | | | | | GAGACAAATAGCTTCCTGGTTCTC | NO. 51 |
| | | | | | AAAACTGTAGCAACGGAAACATTT | |
| | | | | | ACTTATTTATGCGTT | |
| SNP00012 | 6_ | 6 | 25951974 | 25952083 | TGAGCTTTTGGCGCCTCCTGCCAC | SEQ |
| | 25952072 | | | | TGCTCACACAGCACCAGCCTTCTG | ID |
| | | | | | GGTGGAGGGTGAACCTGGCCTGG | NO. 52 |
| | | | | | CTTGTCTGACCTGGAAGCTCCTTG | |
| | | | | | AGGTCAGGAGCCTGT | |
| | | | 25951996 | 25952105 | ACTGCTCACACAGCACCAGCCTTC | SEQ |
| | | | | | TGGGTGGAGGGTGAACCTGGCCT | ID |
| | | | | | GGCTTGTCTGACCTGGAAGCTCCT | NO. 53 |
| | | | | | TGAGGTCAGGAGCCTGTCTTCTTC | |
| | | | | | AGCCCATTATGCCCA | |
| | | | 25952018 | 25952127 | TCTGGGTGGAGGGTGAACCTGGCC | SEQ |
| | | | | | TGGCTTGTCTGACCTGGAAGCTCC | ID |
| | | | | | TTGAGGTCAGGAGCCTGTCTTCTT | NO. 54 |
| | | | | | CAGCCCATTATGCCCAGGTCTAGCT | |
| | | | | | GGGTGCCTCCTGC | |
| | | | 25952040 | 25952149 | CCTGGCTTGTCTGACCTGGAAGCT | SEQ |
| | | | | | CCTTGAGGTCAGGAGCCTGTCTTC | ID |
| | | | | | TTCAGCCCATTATGCCCAGGTCTAG | NO. 55 |
| | | | | | CTGGGTGCCTCCTGCATCTTGGAT | |
| | | | | | GCCGGGTGCTCAG | |
| | | | 25952062 | 25952171 | CTCCTTGAGGTCAGGAGCCTGTCT | SEQ |
| | | | | | TCTTCAGCCCATTATGCCCAGGTCT | ID |
| | | | | | AGCTGGGTGCCTCCTGCATCTTGG | NO. 56 |
| | | | | | ATGCCGGGTGCTCAGGACCACGTG | |
| | | | | | AATGAGAATGTCC | |
| SNP00013 | 6_ | 6 | 371 | 371 | GATTAAAATCTCACCACTACTGATC | SEQ |
| | 37126564 | | 264 | 265 | AGAGAGCTGTAGGCAACTCGCTGC | ID |
| | | | 66 | 75 | ACATGGCATTGAGCTGTGAGGCAC | NO. 57 |
| | | | | | AAGCTTCTGAATAATGGAAGCTCA | |
| | | | | | ATTGTCTCCCAAG | |
| | | | 371 | 371 | ATCAGAGAGCTGTAGGCAACTCGC | SEQ |
| | | | 264 | 265 | TGCACATGGCATTGAGCTGTGAGG | ID |
| | | | 88 | 97 | CACAAGCTTCTGAATAATGGAAGC | NO. 58 |
| | | | | | TCAATTGTCTCCCAAGCCAATTTAG | |
| | | | | | ACCAAGACTTAGC | |
| | | | 371 | 371 | GCTGCACATGGCATTGAGCTGTGA | SEQ |
| | | | 265 | 266 | GGCACAAGCTTCTGAATAATGGAA | ID |
| | | | 10 | 19 | GCTCAATTGTCTCCCAAGCCAATTT | NO. 59 |
| | | | | | AGACCAAGACTTAGCTGAAAGGTA | |
| | | | | 371 | AGGGCAGTCTCTT | |
| | | | 371 | | GAGGCACAAGCTTCTGAATAATGG | SEQ |
| | | | 265 | 266 | AAGCTCAATTGTCTCCCAAGCCAA | ID |
| | | | 32 | 41 | TTTAGACCAAGACTTAGCTGAAAG | NO. 60 |
| | | | | | GTAAGGGCAGTCTCTTCAGGACGC | |
| | | | | | TCTTACTGAGAGAC | |
| | | | 371 | 371 | GGAAGCTCAATTGTCTCCCAAGCC | SEQ |
| | | | 265 | 266 | AATTTAGACCAAGACTTAGCTGAA | ID |
| | | | 54 | 63 | AGGTAAGGGCAGTCTCTTCAGGAC | NO. 61 |
| | | | | | GCTCTTACTGAGAGACCCTCCCTT | |
| | | | | | AATGAGGGCTCCTG | |
| SNP00014 | 10_ | 10 | 517 | 517 | ATGGTCACATTCACACAAATAGTA | SEQ |
| | 51739659 | | 395 | 396 | GCTCATTCTTAGAATGGAAATGAA | ID |
| | | | 61 | 70 | GAATGCAAAAAGTATGTTTTGATTT | NO. 62 |
| | | | | | TTTTGATAACTGACCTTCTCTATAC | |
| | | | | | GTTTGAATTCAC | |
| | | | 517 | 517 | TAGCTCATTCTTAGAATGGAAATGA | SEQ |
| | | | 395 | 396 | AGAATGCAAAAAGTATGTTTTGAT | ID |
| | | | 83 | 92 | TTTTTTGATAACTGACCTTCTCTAT | NO. 63 |
| | | | | | ACGTTTGAATTCACACAACTGAGT | |
| | | | | | TCAGAGTTTGTG | |
| | | | 517 | 517 | TGAAGAATGCAAAAAGTATGTTTT | SEQ |
| | | | 396 | 397 | GATTTTTTTGATAACTGACCTTCTC | ID |
| | | | 05 | 14 | TATACGTTTGAATTCACACAACTGA | NO. 64 |
| | | | | | GTTCAGAGTTTGTGTATGTGTTACA | |
| | | | | | GCTGCATATTG | |
| | | | 517 | 517 | TTGATTTTTTTGATAACTGACCTTC | SEQ |
| | | | 396 | 397 | TCTATACGTTTGAATTCACACAACT | ID |
| | | | 27 | 36 | GAGTTCAGAGTTTGTGTATGTGTTA | NO. 65 |
| | | | | | CAGCTGCATATTGTAAGTAGGGGT | |
| | | | | | AGCTCTTATTT | |
| | | | 517 | 517 | TTCTCTATACGTTTGAATTCACACA | SEQ |
| | | | 396 | 397 | ACTGAGTTCAGAGTTTGTGTATGT | ID |
| | | | 49 | 58 | GTTACAGCTGCATATTGTAAGTAGG | NO. 66 |
| | | | | | GGTAGCTCTTATTTGGTCGAACATA | |
| | | | | | GATTGATTTTC | |
| | | | 891 | 891 | CCTTCCTAACTGTTGTATTCAAAAT | SEQ |
| | | | 754 | 765 | GATTTATGGTTTCAACTTTTAGATA | ID |
| | | | 5 | 4 | TTCCTAAGTCTGTCCTAAAGTTCA | NO. 67 |
| | | | | | AATTAAATAGACCCTTTTCCAAAA | |
| | | | | | CTTCTTACACTG | |
| | | | | | AAAGTTCAAATTAAATAGACCCTT | |
| SNP00015 | 11_ | 11 | 891 | 891 | TTCCAAAACTTCTTACACTGTTTTT | SEQ |
| | 8917643 | | 761 | 772 | TCCAGATATATTCTTTTTGCCTGTCT | ID |
| | | | 1 | 0 | ATTTTTCTAAATAGCAGAGCCTTCC | NO. 68 |
| | | | | | TTTTCACCCA | |
| | | | 891 | 891 | TTTTCCAAAACTTCTTACACTGTTT | SEQ |
| | | | 763 | 774 | TTTCCAGATATATTCTTTTTGCCTGT | ID |
| | | | 3 | 2 | CTATTTTTCTAAATAGCAGAGCCTT | NO. 69 |
| | | | | | CCTTTTCACCCAATTCTTCTTCCAC | |
| | | | | | TTCACTTAT | |
| SNP00016 | 12_ | 12 | 251 | 251 | GCTGAGCACATCTCTCCTTGGCTG | SEQ |
| | 25119445 | | 193 | 194 | CCCCTGACCTCATCTCCCCAGAGG | ID |
| | | | 47 | 56 | CAGTAGGAATTCTCCCCAAAGAAT | NO. 70 |
| | | | | | TCTTCCCCACCTCACTGTTGCGTAC | |
| | | | | | AGGGTCCTCTGCA | |
| | | | 251 | 251 | TGCCCCTGACCTCATCTCCCCAGA | SEQ |
| | | | 193 | 194 | GGCAGTAGGAATTCTCCCCAAAGA | ID |
| | | | 69 | 78 | ATTCTTCCCCACCTCACTGTTGCGT | NO. 71 |
| | | | | | ACAGGGTCCTCTGCAATCTCAGGA | |
| | | | | | AACTCCCGAGGAG | |
| | | | 251 | 251 | GAGGCAGTAGGAATTCTCCCCAAA | SEQ |
| | | | 193 | 195 | GAATTCTTCCCCACCTCACTGTTGC | ID |
| | | | 91 | 00 | GTACAGGGTCCTCTGCAATCTCAG | NO. 72 |
| | | | | | GAAACTCCCGAGGAGTTGACAGG | |
| | | | | | GTTCTTCTGCTCCC | |
| | | | 251 | 251 | AAGAATTCTTCCCCACCTCACTGT | SEQ |
| | | | 194 | 195 | TGCGTACAGGGTCCTCTGCAATCT | ID |
| | | | 13 | 22 | CAGGAAACTCCCGAGGAGTTGAC | NO. 73 |
| | | | | | AGGGTTCTTCTGCTCCCCGTGGGC | |
| | | | | | CTGTGGGTCACAGTA | |
| | | | 251 | 251 | GTTGCGTACAGGGTCCTCTGCAAT | SEQ |
| | | | 194 | 195 | CTCAGGAAACTCCCGAGGAGTTG | ID |
| | | | 35 | 44 | ACAGGGTTCTTCTGCTCCCCGTGG | NO. 74 |
| | | | | | GCCTGTGGGTCACAGTATGACCTT | |
| | | | | | TTGCTTATTTCAGTG | |
| SNP00017 | 12_ | 12 | 251 | 251 | ACTATGAAGATGGAATTAAATAATG | SEQ |
| | 25120732 | | 206 | 207 | CATGGCAAGCTCTGCTTGCCAGAG | ID |
| | | | 34 | 43 | GTTGACACTTAGTAAGGGTTTAAC | NO. 75 |
| | | | | | AGCTGCAAATTGCACATTACCTTT | |
| | | | | | GCGGAGAGCGAGG | |
| | | | 251 | 251 | ATGCATGGCAAGCTCTGCTTGCCA | SEQ |
| | | | 206 | 207 | GAGGTTGACACTTAGTAAGGGTTT | ID |
| | | | 56 | 65 | AACAGCTGCAAATTGCACATTACC | NO. 76 |
| | | | | | TTTGCGGAGAGCGAGGCTTGAGG | |
| | | | | | GGAGGAGGTACATGA | |
| | | | 251 | 251 | CAGAGGTTGACACTTAGTAAGGGT | SEQ |
| | | | 206 | 207 | TTAACAGCTGCAAATTGCACATTA | ID |
| | | | 78 | 87 | CCTTTGCGGAGAGCGAGGCTTGAG | NO. 77 |
| | | | | | GGGAGGAGGTACATGAGGCTGGTT | |
| | | | | | TTGAATACCCCACG | |
| | | | 251 | 251 | GTTTAACAGCTGCAAATTGCACAT | SEQ |
| | | | 207 | 208 | TACCTTTGCGGAGAGCGAGGCTTG | ID |
| | | | 00 | 09 | AGGGGAGGAGGTACATGAGGCTG | NO. 78 |
| | | | | | GTTTTGAATACCCCACGGGGTCAG | |
| | | | | | CTCTAGCACTTGGCT | |
| | | | 251 | 251 | ATTACCTTTGCGGAGAGCGAGGCT | SEQ |
| | | | 207 | 208 | TGAGGGGAGGAGGTACATGAGGC | ID |
| | | | 22 | 31 | TGGTTTTGAATACCCCACGGGGTC | NO. 79 |
| | | | | | AGCTCTAGCACTTGGCTTTGACAA | |
| | | | | | ACACTGAAGCTGTGG | |
| SNP00018 | 12_ | 12 | 251 | 251 | AACCATTGGACTGCCAGGGAAGCC | SEQ |
| | 25135944 | | 358 | 359 | CCTACCACTTAGTTTTAAATTTGAA | ID |
| | | | 46 | 55 | TTGCAGTTGGATTTTCCTAGTGGGT | NO. 80 |
| | | | | | TCTTTCCCTAGTTATAAGAGTCAGA | |
| | | | | | TGCTCTTAACC | |
| | | | 251 | 251 | CCCCTACCACTTAGTTTTAAATTTG | SEQ |
| | | | 358 | 359 | AATTGCAGTTGGATTTTCCTAGTGG | ID |
| | | | 68 | 77 | GTTCTTTCCCTAGTTATAAGAGTCA | NO. 81 |
| | | | | | GATGCTCTTAACCAAAGGAAGTCT | |
| | | | | | TTTGCAAGGTA | |
| | | | 251 | 251 | TTGAATTGCAGTTGGATTTTCCTAG | SEQ |
| | | | 358 | 359 | TGGGTTCTTTCCCTAGTTATAAGAG | ID |
| | | | 90 | 99 | TCAGATGCTCTTAACCAAAGGAAG | NO. 82 |
| | | | | | TCTTTTGCAAGGTATGTTTTTTAAA | |
| | | | | | CAAAACCCAGG | |
| | | | 251 | 251 | TAGTGGGTTCTTTCCCTAGTTATAA | SEQ |
| | | | 359 | 360 | GAGTCAGATGCTCTTAACCAAAGG | ID |
| | | | 12 | 21 | AAGTCTTTTGCAAGGTATGTTTTTT | NO. 83 |
| | | | | | AAACAAAACCCAGGGTGTGGATTC | |
| | | | | | AGACGATTTGCA | |
| | | | 251 | 251 | TAAGAGTCAGATGCTCTTAACCAA | SEQ |
| | | | 359 | 360 | AGGAAGTCTTTTGCAAGGTATGTT | ID |
| | | | 34 | 43 | TTTTAAACAAAACCCAGGGTGTGG | NO. 84 |
| | | | | | ATTCAGACGATTTGCATGTAAGAC | |
| | | | | | CTGTCAGCTGGGTA | |
| SNP00019 | 12_ | 12 | 251 | 251 | CGTGATCTACCTTCTCACCCACAA | SEQ |
| | 25149517 | | 494 | 495 | ACAAGTGGAGTTGGGCAAACACC | ID |
| | | | 19 | 28 | ATCTTTGCTAAAAATGCACACAATT | NO. 85 |
| | | | | | CCAAGCTCCTCAGTCTCTGAAGTA | |
| | | | | | GGCGCTGAGGACTT | |
| | | | 251 | 251 | AAACAAGTGGAGTTGGGCAAACA | SEQ |
| | | | 494 | 495 | CCATCTTTGCTAAAAATGCACACA | ID |
| | | | 41 | 50 | ATTCCAAGCTCCTCAGTCTCTGAA | NO. 86 |
| | | | | | GTAGGCGCTGAGGACTTCAGAGCT | |
| | | | | | GGGACAGAATAACCG | |
| | | | 251 | 251 | ACCATCTTTGCTAAAAATGCACAC | SEQ |
| | | | 494 | 495 | AATTCCAAGCTCCTCAGTCTCTGA | ID |
| | | | 63 | 72 | AGTAGGCGCTGAGGACTTCAGAG | NO. 87 |
| | | | | | CTGGGACAGAATAACCGCCAGAA | |
| | | | | | AAATGCAGATTGAATT | |
| | | | 251 | 251 | ACAATTCCAAGCTCCTCAGTCTCT | SEQ |
| | | | 494 | 495 | GAAGTAGGCGCTGAGGACTTCAG | ID |
| | | | 85 | 94 | AGCTGGGACAGAATAACCGCCAG | NO. 88 |
| | | | | | AAAAATGCAGATTGAATTGTGACA | |
| | | | | | CCCATCCCACCCTACC | |
| | | | 251 | 251 | CTGAAGTAGGCGCTGAGGACTTCA | SEQ |
| | | | 495 | 496 | GAGCTGGGACAGAATAACCGCCA | ID |
| | | | 07 | 16 | GAAAAATGCAGATTGAATTGTGAC | NO. 89 |
| | | | | | ACCCATCCCACCCTACCCCCACCA | |
| | | | | | CTCACAGGCTAGGAA | |
| SNP00020 | 12_ | 12 | 251 | 251 | GTAGGGCGGGGCCTTGCGCTCAGC | SEQ |
| | 251 | | 524 | 525 | TCCTAGGATCCTCGAAGCTGGCCT | ID |
| | 52554 | | 56 | 65 | GGTATTTAGCCTGTGGTCCCCACTG | NO. 90 |
| | | | | | ACAAGGAAATGGGGACGTGACCC | |
| | | | | | AGTGCATTGGAATG | |
| | | | 251 | 251 | GCTCCTAGGATCCTCGAAGCTGGC | SEQ |
| | | | 524 | 525 | CTGGTATTTAGCCTGTGGTCCCCAC | ID |
| | | | 78 | 87 | TGACAAGGAAATGGGGACGTGAC | NO. 91 |
| | | | | | CCAGTGCATTGGAATGACACGGTC | |
| | | | | | CTGTGTCAACCTCA | |
| | | | 251 | 251 | GCCTGGTATTTAGCCTGTGGTCCCC | SEQ |
| | | | 525 | 526 | ACTGACAAGGAAATGGGGACGTG | ID |
| | | | 00 | 09 | ACCCAGTGCATTGGAATGACACGG | NO. 92 |
| | | | | | TCCTGTGTCAACCTCAGTCCCCTC | |
| | | | | | CCGGGGAAACCTCA | |
| | | | 251 | 251 | CCCACTGACAAGGAAATGGGGAC | SEQ |
| | | | 525 | 526 | GTGACCCAGTGCATTGGAATGACA | ID |
| | | | 22 | 31 | CGGTCCTGTGTCAACCTCAGTCCC | NO. 93 |
| | | | | | CTCCCGGGGAAACCTCAACCACCT | |
| | | | | | CCCACCCCACCAACA | |
| | | | 251 | 251 | CGTGACCCAGTGCATTGGAATGAC | SEQ |
| | | | 525 | 526 | ACGGTCCTGTGTCAACCTCAGTCC | ID |
| | | | 44 | 53 | CCTCCCGGGGAAACCTCAACCACC | NO. 94 |
| | | | | | TCCCACCCCACCAACAACCCTGCC | |
| | | | | | ATCTCTTGGGGAGC | |
| SNP00021 | 12_ | 12 | 251 | 251 | GCGAACATAACTGAGGTTTCCGCT | SEQ |
| | 25154575 | | 544 | 545 | GAGATTCCTTGCCAAGGCCTCCCT | ID |
| | | | 77 | 86 | AGAATATTTGCGCATCAGTCTCCG | NO. 95 |
| | | | | | GGGGCTTCCGCTGTCTGGTCAGCT | |
| | | | | | CTCGATGAGATCCT | |
| | | | 251 | 251 | CTGAGATTCCTTGCCAAGGCCTCC | SEQ |
| | | | 544 | 546 | CTAGAATATTTGCGCATCAGTCTCC | ID |
| | | | 99 | 08 | GGGGGCTTCCGCTGTCTGGTCAGC | NO. 96 |
| | | | | | TCTCGATGAGATCCTCCTACCTGA | |
| | | | | | GGACTCCAGGCCT | |
| | | | 251 | 251 | CCCTAGAATATTTGCGCATCAGTCT | SEQ |
| | | | 545 | 546 | CCGGGGGCTTCCGCTGTCTGGTCA | ID |
| | | | 21 | 30 | GCTCTCGATGAGATCCTCCTACCTG | NO. 97 |
| | | | | | AGGACTCCAGGCCTCTCCTGTGGC | |
| | | | | | TGTGTCGTTTGC | |
| | | | 251 | 251 | TCTCCGGGGGCTTCCGCTGTCTGG | SEQ |
| | | | 545 | 546 | TCAGCTCTCGATGAGATCCTCCTAC | ID |
| | | | 43 | 52 | CTGAGGACTCCAGGCCTCTCCTGT | NO. 98 |
| | | | | | GGCTGTGTCGTTTGCTCTCTGCCA | |
| | | | | | CTGTGACCGAGGT | |
| | | | 251 | 251 | GGTCAGCTCTCGATGAGATCCTCC | SEQ |
| | | | 545 | 546 | TACCTGAGGACTCCAGGCCTCTCC | ID |
| | | | 65 | 74 | TGTGGCTGTGTCGTTTGCTCTCTGC | NO. 99 |
| | | | | | CACTGTGACCGAGGTGTGTAACTC | |
| | | | | | GGCCCCTTCTCCT | |
| SNP00022 | 12_ | 12 | 251 | 251 | GGGAGGGGAAGGCATGCTGTGTG | SEQ |
| | 25155325 | | 552 | 553 | AGAGGAATAGTGTAAGGAACTGA | ID |
| | | | 27 | 36 | GTCTGAAGACCACAAACATGGATA | NO. 100 |
| | | | | | GAGTGGAGGATTTATTCTTTACCTT | |
| | | | | | CTCTACAGTAAGGCA | |
| | | | 251 | 251 | GAGAGGAATAGTGTAAGGAACTG | SEQ |
| | | | 552 | 553 | AGTCTGAAGACCACAAACATGGAT | ID |
| | | | 49 | 58 | AGAGTGGAGGATTTATTCTTTACCT | NO. 101 |
| | | | | | TCTCTACAGTAAGGCAATGGTGAA | |
| | | | 251 | 251 | TGAACTCTGCCTGT | |
| | | | 552 | 553 | GAGTCTGAAGACCACAAACATGG | SEQ |
| | | | 71 | 80 | ATAGAGTGGAGGATTTATTCTTTAC | ID |
| | | | | | CTTCTCTACAGTAAGGCAATGGTG | NO. 102 |
| | | | | | AATGAACTCTGCCTGTTAATGTTGC | |
| | | | 251 | 251 | ACATTGAAACACT | |
| | | | 552 | 554 | GATAGAGTGGAGGATTTATTCTTTA | SEQ |
| | | | 93 | 02 | CCTTCTCTACAGTAAGGCAATGGT | ID |
| | | | | | GAATGAACTCTGCCTGTTAATGTT | NO. 103 |
| | | | | | GCACATTGAAACACTAACTAAAAT | |
| | | | 251 | 251 | TCGGGCTGCGGCT | |
| | | | 553 | 554 | TTACCTTCTCTACAGTAAGGCAATG | SEQ |
| | | | 15 | 24 | GTGAATGAACTCTGCCTGTTAATG | ID |
| | | | | | TTGCACATTGAAACACTAACTAAA | NO. 104 |
| | | | | | ATTCGGGCTGCGGCTGTGCTACTC | |
| | | | | | ATAGTGCCATATG | |
| SNP00023 | 12_ | 12 | 362 | 362 | AGAGAGCAGAAGCGAGCAGCTCT | SEQ |
| | 36292909 | | 928 | 929 | GCGCTTTATAACCCAGCTCTGCACT | ID |
| | | | 11 | 20 | TTATAACCCAGCTTTTCATTAAAGG | NO. 105 |
| | | | | | AGCTATTGGCAACCCTGGAAAGAG | |
| | | | | | TGGGGGAAGCAGA | |
| | | | 362 | 362 | TGCGCTTTATAACCCAGCTCTGCAC | SEQ |
| | | | 928 | 929 | TTTATAACCCAGCTTTTCATTAAAG | ID |
| | | | 33 | 42 | GAGCTATTGGCAACCCTGGAAAGA | NO. 106 |
| | | | | | GTGGGGGAAGCAGAGGGGAAGGA | |
| | | | | | GAGAAGTGGGGAA | |
| | | | 362 | 362 | CACTTTATAACCCAGCTTTTCATTA | SEQ |
| | | | 928 | 929 | AAGGAGCTATTGGCAACCCTGGAA | ID |
| | | | 55 | 64 | AGAGTGGGGGAAGCAGAGGGGAA | NO. 107 |
| | | | | | GGAGAGAAGTGGGGAAGAGAATT | |
| | | | | | TCTCGTTCAAATAAT | |
| | | | 362 | 362 | TTAAAGGAGCTATTGGCAACCCTG | SEQ |
| | | | 928 | 929 | GAAAGAGTGGGGGAAGCAGAGGG | ID |
| | | | 77 | 86 | GAAGGAGAGAAGTGGGGAAGAGA | NO. 108 |
| | | | | | ATTTCTCGTTCAAATAATCCCATGG | |
| | | | | | TTTTCAGCAAGGTCA | |
| | | | 362 | 362 | TGGAAAGAGTGGGGGAAGCAGAG | SEQ |
| | | | 928 | 930 | GGGAAGGAGAGAAGTGGGGAAGA | ID |
| | | | 99 | 08 | GAATTTCTCGTTCAAATAATCCCAT | NO. 109 |
| | | | | | GGTTTTCAGCAAGGTCAAAAGTCT | |
| | | | | | GAGAGTTCTGTGGTG | |
| SNP00024 | 12_ | 12 | 785 | 785 | GGTTTGAACTGGAGTATACAGGTG | SEQ |
| | 78576808 | | 767 | 768 | AATCACGATCTTGTGGGGCTTTTTT | ID |
| | | | 10 | 19 | CCTTTTCAGCCCAATGCAACTTGAT | NO. 110 |
| | | | | | TTTCTTGGTGACTGGAAGTTTCTTT | |
| | | | | | CCTAAATCTGT | |
| | | | 785 | 785 | AGTATACAGGTGAATCACGATCTT | SEQ |
| | | | 767 | 768 | GTGGGGCTTTTTTCCTTTTCAGCCC | ID |
| | | | 22 | 31 | AATGCAACTTGATTTTCTTGGTGAC | NO. 111 |
| | | | | | TGGAAGTTTCTTTCCTAAATCTGTG | |
| | | | | | AAGCTGTATTT | |
| | | | 785 | 785 | TTGTGGGGCTTTTTTCCTTTTCAGC | SEQ |
| | | | 767 | 768 | CCAATGCAACTTGATTTTCTTGGTG | ID |
| | | | 44 | 53 | ACTGGAAGTTTCTTTCCTAAATCTG | NO. 112 |
| | | | | | TGAAGCTGTATTTCTTTGGGAAGG | |
| | | | | | AGCTTCACCTG | |
| | | | 785 | 785 | AGCCCAATGCAACTTGATTTTCTTG | SEQ |
| | | | 767 | 768 | GTGACTGGAAGTTTCTTTCCTAAA | ID |
| | | | 66 | 75 | TCTGTGAAGCTGTATTTCTTTGGGA | NO. 113 |
| | | | | | AGGAGCTTCACCTGTGTCTATTTTT | |
| | | | | | AGGTTACACCT | |
| | | | 785 | 785 | TCCTAAATCTGTGAAGCTGTATTTC | SEQ |
| | | | 768 | 769 | TTTGGGAAGGAGCTTCACCTGTGT | ID |
| | | | 08 | 17 | CTATTTTTAGGTTACACCTAGAGGT | NO. 114 |
| | | | | | GATATCACATGATGCGTGTGTCTCC | |
| | | | | | CTTACTTCACT | |
| SNP00025 | 15_ | 15 | 687 | 687 | TTGTTAGTGTCATAGAATTAAAGGC | SEQ |
| | 68745093 | | 449 | 45 | TGCTGGGAGGAAGATCAACAGTTG | ID |
| | | | 95 | 04 | AAAACCCTAAAAAAAAGTGATAGC | NO. 115 |
| | | | | | TGATGTTTCTCATTTCTTTCTTCAA | |
| | | | | | CGTAAGTATATG | |
| | | | 687 | 687 | GGCTGCTGGGAGGAAGATCAACA | SEQ |
| | | | 450 | 45 | GTTGAAAACCCTAAAAAAAAGTG | ID |
| | | | 17 | 26 | ATAGCTGATGTTTCTCATTTCTTTC | NO. 116 |
| | | | | | TTCAACGTAAGTATATGAATTTTAA | |
| | | | | | AAAACTTTTAAATT | |
| SNP00026 | 20_ | 20 | 251 | 251 | TAAGCCTGCGTGGAACGCAGAGC | SEQ |
| | 25185724 | | 856 | 857 | CTGCGCAATAGCACCGGTGAAGTT | ID |
| | | | 26 | 35 | CCCGATGCCCTGAACTCTGTTTCTC | NO. 117 |
| | | | | | TCAGTGCCAGGCCTGGCAGGGTTG | |
| | | | | | TGCGGCTGTGTTTG | |
| | | | 251 | 251 | CCTGCGCAATAGCACCGGTGAAGT | SEQ |
| | | | 856 | 857 | TCCCGATGCCCTGAACTCTGTTTCT | ID |
| | | | 48 | 57 | CTCAGTGCCAGGCCTGGCAGGGTT | NO. 118 |
| | | | | | GTGCGGCTGTGTTTGGACGGCAGA | |
| | | | | | CGCCCACTGTGGT | |
| | | | 251 | 251 | GTTCCCGATGCCCTGAACTCTGTTT | SEQ |
| | | | 856 | 857 | CTCTCAGTGCCAGGCCTGGCAGGG | ID |
| | | | 70 | 79 | TTGTGCGGCTGTGTTTGGACGGCA | NO. 119 |
| | | | | | GACGCCCACTGTGGTGCTGCCTCC | |
| | | | | | TCGCGGGTTCTGG | |
| | | | 251 | 251 | TTTCTCTCAGTGCCAGGCCTGGCA | SEQ |
| | | | 856 | 858 | GGGTTGTGCGGCTGTGTTTGGACG | ID |
| | | | 92 | 01 | GCAGACGCCCACTGTGGTGCTGCC | NO. 120 |
| | | | | | TCCTCGCGGGTTCTGGAAGGGAGC | |
| | | | | | CAGTTCTTGGTTTT | |
| | | | 251 | 251 | CAGGGTTGTGCGGCTGTGTTTGGA | SEQ |
| | | | 857 | 858 | CGGCAGACGCCCACTGTGGTGCTG | ID |
| | | | 14 | 23 | CCTCCTCGCGGGTTCTGGAAGGGA | NO. 121 |
| | | | | | GCCAGTTCTTGGTTTTAGATGAAA | |
| | | | | | GGGTTAAATGCCTA | |
| SNP00027 | 20_ | 20 | 378 | 378 | CTATGACAAGGACCATGCTTTGCC | SEQ |
| | 37863763 | | 636 | 637 | TTGTCCCTATGCCTTCACACCTGCT | ID |
| | | | 65 | 74 | GTTCCATCAAACTATAATGCTCCTC | NO. 122 |
| | | | | | CCCTGCTTTTCATCTGAGTCAATCC | |
| | | | | | CCTTTCATTTG | |
| | | | 378 | 378 | CCTTGTCCCTATGCCTTCACACCTG | SEQ |
| | | | 636 | 637 | CTGTTCCATCAAACTATAATGCTCC | ID |
| | | | 87 | 96 | TCCCCTGCTTTTCATCTGAGTCAAT | NO. 123 |
| | | | | | CCCCTTTCATTTGAGATGCAAACC | |
| | | | | | AAGTGTCAAAT | |
| | | | 378 | 378 | CTGCTGTTCCATCAAACTATAATGC | SEQ |
| | | | 637 | 638 | TCCTCCCCTGCTTTTCATCTGAGTC | ID |
| | | | 09 | 18 | AATCCCCTTTCATTTGAGATGCAAA | NO. 124 |
| | | | | | CCAAGTGTCAAATATCATCTCATTC | |
| | | | | | CGTAAGCTCT | |
| | | | 378 | 378 | TGCTCCTCCCCTGCTTTTCATCTGA | SEQ |
| | | | 637 | 638 | GTCAATCCCCTTTCATTTGAGATGC | ID |
| | | | 31 | 40 | AAACCAAGTGTCAAATATCATCTC | NO. 125 |
| | | | | | ATTCCGTAAGCTCTCATATACCCTA | |
| | | | | | ACTCACTCCAG | |
| | | | 378 | 378 | TGAGTCAATCCCCTTTCATTTGAGA | SEQ |
| | | | 637 | 638 | TGCAAACCAAGTGTCAAATATCAT | ID |
| | | | 53 | 62 | CTCATTCCGTAAGCTCTCATATACC | NO. 126 |
| | | | | | CTAACTCACTCCAGTAATCTTGCCT | |
| | | | | | GGGAAATCCCA | |
| SNP00028 | 20_ | 20 | 441 | 441 | CTTCAGTCAAGGCAGACGGCTGG | SEQ |
| | 44119346 | | 192 | 193 | AAAACACTGATCAAAGGGGAAGC | ID |
| | | | 48 | 57 | TTGGTTAGTTTACACTCTCCAGCTC | NO. 127 |
| | | | | | GATTTGGATAGAGGCTTCAGGATG | |
| | | | | | CTTGCTTCTGACTTG | |
| | | | 441 | 441 | GAAAACACTGATCAAAGGGGAAG | SEQ |
| | | | 192 | 193 | CTTGGTTAGTTTACACTCTCCAGCT | ID |
| | | | 70 | 79 | CGATTTGGATAGAGGCTTCAGGAT | NO. 128 |
| | | | | | GCTTGCTTCTGACTTGATCTGTAAT | |
| | | | | | CCTAGGGAAGCTT | |
| | | | 441 | 441 | GCTTGGTTAGTTTACACTCTCCAGC | SEQ |
| | | | 192 | 194 | TCGATTTGGATAGAGGCTTCAGGA | ID |
| | | | 92 | 01 | TGCTTGCTTCTGACTTGATCTGTAA | NO. 129 |
| | | | | | TCCTAGGGAAGCTTCATGAAACAC | |
| | | | | | TTGATAGCCTTA | |
| | | | 441 | 441 | AGCTCGATTTGGATAGAGGCTTCA | SEQ |
| | | | 193 | 194 | GGATGCTTGCTTCTGACTTGATCTG | ID |
| | | | 14 | 23 | TAATCCTAGGGAAGCTTCATGAAA | NO. 130 |
| | | | | | CACTTGATAGCCTTAAACCATTCAA | |
| | | | | | GGGCTGGTTAAA | |
| | | | 441 | 441 | CAGGATGCTTGCTTCTGACTTGATC | SEQ |
| | | | 193 | 194 | TGTAATCCTAGGGAAGCTTCATGA | ID |
| | | | 36 | 45 | AACACTTGATAGCCTTAAACCATTC | NO. 131 |
| | | | | | AAGGGCTGGTTAAAAAAAATAAAT | |
| | | | | | ACAGAGTTATGA | |
| SNP00029 | 22_ | 22 | 469 | 469 | GGCACTCCCTGGGCCAGCTTCCCC | SEQ |
| | 46988971 | | 888 | 889 | GCTCTGAGCAGGGCAGGGTGGAG | ID |
| | | | 73 | 82 | GGTGAGCCAGCCGGGAAGGGGTT | NO. 132 |
| | | | | | GCCAGTCTGCAGAGACTGAGAGC | |
| | | | | | CACGCGTGCTCCTGGGC | |
| | | | 469 | 469 | CCGCTCTGAGCAGGGCAGGGTGG | SEQ |
| | | | 888 | 890 | AGGGTGAGCCAGCCGGGAAGGGG | ID |
| | | | 95 | 04 | TTGCCAGTCTGCAGAGACTGAGAG | NO. 133 |
| | | | | | CCACGCGTGCTCCTGGGCAGGTGA | |
| | | | | | GCTTCATTCCCATCCT | |
| | | | 469 | 469 | GAGGGTGAGCCAGCCGGGAAGGG | SEQ |
| | | | 889 | 890 | GTTGCCAGTCTGCAGAGACTGAGA | ID |
| | | | 17 | 26 | GCCACGCGTGCTCCTGGGCAGGTG | NO. 134 |
| | | | | | AGCTTCATTCCCATCCTGAGGGCT | |
| | | | | | GGGCCGGGGAGCTGG | |
| | | | 469 | 469 | GGTTGCCAGTCTGCAGAGACTGAG | SEQ |
| | | | 889 | 890 | AGCCACGCGTGCTCCTGGGCAGGT | ID |
| | | | 39 | 48 | GAGCTTCATTCCCATCCTGAGGGC | NO. 135 |
| | | | | | TGGGCCGGGGAGCTGGGCCTGGG | |
| | | | | | AGCTGGCCCTGGGTT | |
| | | | 469 | 469 | GCAGAGACTGAGAGCCACGCGTG | SEQ |
| | | | 889 | 890 | CTCCTGGGCAGGTGAGCTTCATTC | ID |
| | | | 51 | 60 | CCATCCTGAGGGCTGGGCCGGGGA | NO. 136 |
| | | | | | GCTGGGCCTGGGAGCTGGCCCTGG | |
| | | | | | GTTTCCCCTGCCTGG | |
| SNP00030 | 22_ | 22 | 470 | 470 | CTGAGAAAGGGACTGGAGATGAG | SEQ |
| | 47002481 | | 023 | 024 | AGGGAAGGAGGGGATAGGAGGGA | ID |
| | | | 83 | 92 | TCTGACGTGGTGTTTCTACTTCTGA | NO. 137 |
| | | | | | GCACTTTGCAGATAAACTGTCCTT | |
| | | | | | AAGAAGGAGCTTCTC | |
| | | | 470 | 470 | GAGGGAAGGAGGGGATAGGAGGG | SEQ |
| | | | 024 | 025 | ATCTGACGTGGTGTTTCTACTTCTG | ID |
| | | | 05 | 14 | AGCACTTTGCAGATAAACTGTCCT | NO. 138 |
| | | | | | TAAGAAGGAGCTTCTCAGTGGAG | |
| | | | | | AGCAAAATGTACTCA | |
| | | | 470 | 470 | GATCTGACGTGGTGTTTCTACTTCT | SEQ |
| | | | 024 | 025 | GAGCACTTTGCAGATAAACTGTCC | ID |
| | | | 27 | 36 | TTAAGAAGGAGCTTCTCAGTGGAG | NO. 139 |
| | | | | | AGCAAAATGTACTCAGACCTCTGA | |
| | | | | | ATGTTGCAGGGCA | |
| | | | 470 | 470 | TCTGAGCACTTTGCAGATAAACTG | SEQ |
| | | | 024 | 025 | TCCTTAAGAAGGAGCTTCTCAGTG | ID |
| | | | 49 | 58 | GAGAGCAAAATGTACTCAGACCTC | NO. 140 |
| | | | | | TGAATGTTGCAGGGCAAATGTTAC | |
| | | | | | TCAAAGTCTAATAA | |
| | | | 470 | 470 | TGTCCTTAAGAAGGAGCTTCTCAG | SEQ |
| | | | 024 | 025 | TGGAGAGCAAAATGTACTCAGACC | ID |
| | | | 71 | 80 | TCTGAATGTTGCAGGGCAAATGTT | NO. 141 |
| | | | | | ACTCAAAGTCTAATAATTTCTTTCA | |
| | | | | | AAGATTTGGATCT | |
| SNP00031 | 22_ | 22 | 502 | 502 | TCAGAGCAGGGGGCCCTGCCGCA | SEQ |
| | 50203275 | | 031 | 032 | GTGGGGAAGGGGGGTCTGCACCA | ID |
| | | | 67 | 76 | GCCACCGCGTCTCACCTGGCCTGC | NO. 142 |
| | | | | | TCCACGTCGCAGATGTAGGCCTGC | |
| | | | | | CCCACCACCTCCTCGT | |
| | | | 502 | 502 | GGGGGTCTGCACCAGCCACCGCGT | SEQ |
| | | | 031 | 033 | CTCACCTGGCCTGCTCCACGTCGC | ID |
| | | | 99 | 08 | AGATGTAGGCCTGCCCCACCACCT | NO. 143 |
| | | | | | CCTCGTGGTACGTGGCTGCCTCCC | |
| | | | | | TCAACCTCAACTCG | |
| | | | 502 | 502 | GTCTCACCTGGCCTGCTCCACGTC | SEQ |
| | | | 032 | 033 | GCAGATGTAGGCCTGCCCCACCAC | ID |
| | | | 21 | 30 | CTCCTCGTGGTACGTGGCTGCCTC | NO. 144 |
| | | | | | CCTCAACCTCAACTCGGAACAAGC | |
| | | | | | CAGGGCAAGTCTGG | |
| | | | 502 | 502 | TCGCAGATGTAGGCCTGCCCCACC | SEQ |
| | | | 032 | 033 | ACCTCCTCGTGGTACGTGGCTGCC | ID |
| | | | 43 | 52 | TCCCTCAACCTCAACTCGGAACAA | NO. 145 |
| | | | | | GCCAGGGCAAGTCTGGGGATGAG | |
| | | | | | AGGAGCGTCAGGCCT | |
| | | | 502 | 502 | CCACCTCCTCGTGGTACGTGGCTG | SEQ |
| | | | 032 | 033 | CCTCCCTCAACCTCAACTCGGAAC | ID |
| | | | 65 | 74 | AAGCCAGGGCAAGTCTGGGGATG | NO. 146 |
| | | | | | AGAGGAGCGTCAGGCCTGAGTCA | |
| | | | | | GGCTGCCCTGGGCTCA | |
| SNP00032 | 25_ | 25 | 137 | 137 | TGGGGTGACAAAGAGCTGGACAC | SEQ |
| | 13791395 | | 912 | 914 | GACTGAAGCGCCTTACCACGCACA | ID |
| | | | 97 | 06 | CATGCTCATGGGAGAATCAGGTAG | NO. 147 |
| | | | | | ACAGCGCCTTGAAGACACTTAGGG | |
| | | | | | GAGTGGTCAGTTCTG | |
| | | | 137 | 137 | CGACTGAAGCGCCTTACCACGCAC | SEQ |
| | | | 913 | 914 | ACATGCTCATGGGAGAATCAGGTA | ID |
| | | | 19 | 28 | GACAGCGCCTTGAAGACACTTAGG | NO. 148 |
| | | | | | GGAGTGGTCAGTTCTGCCTTTGGG | |
| | | | | | ATGGTGATAGACAG | |
| | | | 137 | 137 | ACACATGCTCATGGGAGAATCAGG | SEQ |
| | | | 913 | 914 | TAGACAGCGCCTTGAAGACACTTA | ID |
| | | | 41 | 50 | GGGGAGTGGTCAGTTCTGCCTTTG | NO. 149 |
| | | | | | GGATGGTGATAGACAGGAAAGTCT | |
| | | | | | CCCCTGAGGAACTG | |
| | | | 137 | 137 | GGTAGACAGCGCCTTGAAGACACT | SEQ |
| | | | 913 | 914 | TAGGGGAGTGGTCAGTTCTGCCTT | ID |
| | | | 63 | 72 | TGGGATGGTGATAGACAGGAAAGT | NO. 150 |
| | | | | | CTCCCCTGAGGAACTGACATTTGA | |
| | | | | | GTCTTAAAAATGTG | |
| | | | 137 | 137 | CTTAGGGGAGTGGTCAGTTCTGCC | SEQ |
| | | | 913 | 914 | TTTGGGATGGTGATAGACAGGAAA | ID |
| | | | 85 | 94 | GTCTCCCCTGAGGAACTGACATTT | NO. 151 |
| | | | | | GAGTCTTAAAAATGTGAATATGCA | |
| | | | | | GATCAATCAAGCGG | |
| SNP00033 | 26_ | 26 | 406 | 406 | CAGCTGCTGAAGTGCACACCCCGA | SEQ |
| | 40609491 | | 093 | 095 | GAAGCCACCGCTCAGCAGCCAAG | ID |
| | | | 93 | 02 | ACGGGCCTCCACTCGGCCAACTGG | NO. 152 |
| | | | | | AGAAAGCCCACACAGAAGCAACG | |
| | | | | | AAGACGCAGCACAGCC | |
| | | | 406 | 406 | GAGAAGCCACCGCTCAGCAGCCA | SEQ |
| | | | 094 | 095 | AGACGGGCCTCCACTCGGCCAACT | ID |
| | | | 15 | 24 | GGAGAAAGCCCACACAGAAGCAA | NO. 153 |
| | | | | | CGAAGACGCAGCACAGCCAAGGA | |
| | | | | | CGCGCCGTTCCTGATTC | |
| | | | 406 | 406 | AAGACGGGCCTCCACTCGGCCAA | SEQ |
| | | | 094 | 095 | CTGGAGAAAGCCCACACAGAAGC | ID |
| | | | 37 | 46 | AACGAAGACGCAGCACAGCCAAG | NO. 154 |
| | | | | | GACGCGCCGTTCCTGATTCTTTTCC | |
| | | | | | TGACGGTGCCTGCTAC | |
| | | | 406 | 406 | ACTGGAGAAAGCCCACACAGAAG | SEQ |
| | | | 094 | 095 | CAACGAAGACGCAGCACAGCCAA | ID |
| | | | 59 | 68 | GGACGCGCCGTTCCTGATTCTTTTC | NO. 155 |
| | | | | | CTGACGGTGCCTGCTACTCTTAGC | |
| | | | | | ATGTTATTGCAAAGT | |
| | | | 406 | 406 | GCAACGAAGACGCAGCACAGCCA | SEQ |
| | | | 094 | 095 | AGGACGCGCCGTTCCTGATTCTTT | ID |
| | | | 81 | 90 | TCCTGACGGTGCCTGCTACTCTTA | NO. 156 |
| | | | | | GCATGTTATTGCAAAGTCATTCACA | |
| | | | | | TCCACCTCTTTTAA | |

In a second aspect, the present disclosure provides a use of a reagent for detecting the combination of 33 SNP locis associated with a wool fiber diameter of fine wool sheep described in the first aspect in detection of a wool fiber diameter of fine wool sheep.

Preferably, the reagent includes primers for detecting the combination of SNP locis, where the primers can be designed by those skilled in the art according to sequence information of each locus in the combination of SNP locis associated with a wool fiber diameter of fine wool sheep provided by the present disclosure, and can allow a detection purpose under the same reaction conditions. The primers are designed by a conventional method according to information of loci in the combination of SNP locis associated with a wool fiber diameter of fine wool sheep provided by the present disclosure without creative efforts. Therefore, the primers obtained according to the combination of SNP locis associated with wool fiber diameter of fine wool sheep provided by the present disclosure also fall within the protection scope of the present disclosure.

Preferably, the reagent includes a molecular probe combination for detecting the combination of SNP locis. Molecular probes are designed by a conventional method according to information of loci in the combination of SNP locis associated with a wool fiber diameter of fine wool sheep provided by the present disclosure without creative efforts. Therefore, the molecular probes obtained according to the combination of biological SNP locis associated with a wool fiber diameter of fine wool sheep provided by the present disclosure also fall within the protection scope of the present disclosure.

Preferably, the molecular probes are shown in Table 1.

Table 1 Molecular probes for the combination of SNP locis associated with a wool fiber diameter of fine wool sheep

Preferably, the reagent includes a gene chip, and the gene chip is prepared by a conventional method as follows: immobilizing the primers or probes on a polymer substrate, such as a nylon membrane, a nitrocellulose (NC) membrane, a plastic, a silica gel wafer, or a magnetic microbead; or immobilizing the probes on a glass plate; or directly synthesizing the primers or probes on a hard surface such as a glass. The SNP gene chip in the present disclosure is used by a conventional method.

In a third aspect, the present disclosure provides a molecular probe combination for analyzing a wool fiber diameter trait of fine wool sheep, where the molecular probe combination is provided to detect the combination of 33 SNP locis associated with a wool fiber diameter trait of fine wool sheep described in the first aspect.

Preferably, the molecular probe combination is shown in Table 1 above.

In a fourth aspect, the present disclosure provides a gene chip for analyzing a wool fiber diameter trait of fine wool sheep, where the gene chip is loaded with the molecular probe combination for analyzing a wool fiber diameter trait of fine wool sheep described in the third aspect.

In a fifth aspect, the present disclosure provides a kit for analyzing a wool fiber diameter trait of fine wool sheep, including the molecular probe combination for analyzing a wool fiber diameter trait of fine wool sheep described in the third aspect or the gene chip for analyzing a wool fiber diameter trait of fine wool sheep described in the fourth aspect.

In a sixth aspect, the present disclosure provides a use of the molecular probe combination described in the third aspect, the gene chip described in the fourth aspect, or the kit described in the fifth aspect in evaluation of a wool fiber diameter trait of fine wool sheep, screening of a fine wool sheep breed, identification of a fine wool sheep breed, or molecular marker-assisted selection of fine wool sheep.

In a seventh aspect, the present disclosure provides a method for analyzing a wool fiber diameter trait of fine wool sheep, including: detecting genotypes of the 33 SNP locis associated with a wool fiber diameter of fine wool sheep described in the first aspect in genomic DNA of the fine wool sheep to be tested; and with reference to genotypes of the 33 SNP locis in genomic DNA of control fine wool sheep, determining the wool fiber diameter trait of the fine wool sheep according to detection results of the genotypes.

Beneficial effects of the present disclosure: The present disclosure provides a combination of 33 SNP locis associated with wool fiber diameter of fine wool sheep, and the SNP locis are determined based on alignment with an Ovis aries reference genome versions 4.0 sequence. The present disclosure discovers that the detection of genotypes for the combination of 33 SNP locis associated with a wool fiber diameter trait of fine wool sheep in genomic DNA of fine wool sheep to be tested by means of molecular probes or a gene chip can be used for analysis of the wool fiber diameter trait of fine wool sheep and early selective breeding of fine wool sheep to allow individual selection for the wool fiber diameter trait that is difficult to measure at an early stage, which can shorten the generation interval, accelerate the breeding process, and largely save the breeding cost, and provides a support for the identification, breed preservation, and genetic breeding of fine wool sheep in the future. Moreover, the molecular probe combination, the gene chip, and the kit produced based on the combination of 33 SNP locis associated with the wool fiber diameter trait of fine wool sheep provided by the present disclosure have a small throughput, a low cost, easy analysis, wide universality, and a promising market prospect compared with the existing high-density chips.

### BRIEF DESCRIPTION OF THE DRAWINGS

FIG. 1 is a Manhattan plot obtained with a P-value of −log 10 under a general linear model (GLM) during genome-wide association study (GWAS) for SNP data associated with the wool fiber diameter trait of fine wool sheep in Example 1, where FD represents the diameter of the wool fiber; and

FIG. 2 is a Q-Q plot obtained with a P-value of −log 10 under GLM during GWAS for SNP data associated with the wool fiber diameter trait of fine wool sheep in Example 1.

### DETAILED DESCRIPTION OF THE EMBODIMENTS

The technical solutions of the present disclosure will be described in detail below with reference to examples. It should be noted that the following examples are provided merely for a purpose of illustration, and are not intended to limit the scope of the present disclosure. Those skilled in the art can make various modifications and substitutions to the present disclosure without departing from the purpose and spirit of the present disclosure.

Unless otherwise specified, all experimental methods for the experiments in the following examples are conventional methods.

Unless otherwise specified, experimental conditions for all experiments in the following examples are conventional conditions, such as conditions recommended by the Molecular Cloning: Experiment Guide of Sambrook et al. or instructions of a manufacturer.

The SNP locis of the present disclosure refers to a DNA sequence polymorphism caused by a variation of a single nucleotide at a genome level.

### Example 1 SNP Locis Associated with a Wool Fiber Diameter Trait of Fine Wool Sheep

### 1. Acquisition of a Total SNP Set

460 fine wool sheep individuals of four representative fine wool sheep breeds in China were subjected to whole-genome resequencing, with an average depth of 5 x, and a resequencing analysis process was used to align resequencing results with the Ovis aries reference genome version 4.0 sequence (obtained from the National Center for Biotechnology Information (NCBI)) released in 2015 by two methods. Common results of alignment by the two methods constituted a SNP locis set.

Specifically, the high-depth resequencing for the plurality of fine wool sheep individuals was completed by a biological sequencing company, and sequencing results produced by the biological sequencing company all could allow the technical purpose of the present disclosure, which was not limited in the present disclosure. In the present disclosure, a Fastq file of a sample returned by the sequencing company was aligned with the Ovis aries reference genome version 4.0 sequence through a BAM file to obtain a BAM file of the sample, and the BAM file of the sample was analyzed with SAMtools and GATK software to obtain a VCF file with population SNP typing information. VCF file results obtained by the two methods were combined and subjected to quality screening to obtain a SNP locis set with 33 SNP locis.

Specifically, the fine wool sheep breeds used in the present disclosure were the following four representative fine wool sheep breeds in China: Chinese Merino sheep, Alpine Merino sheep, Aohan fine wool sheep, and Qinghai fine wool sheep.

2. Screening of Candidate Genes and Functional Regions where the Candidate Genes are Located

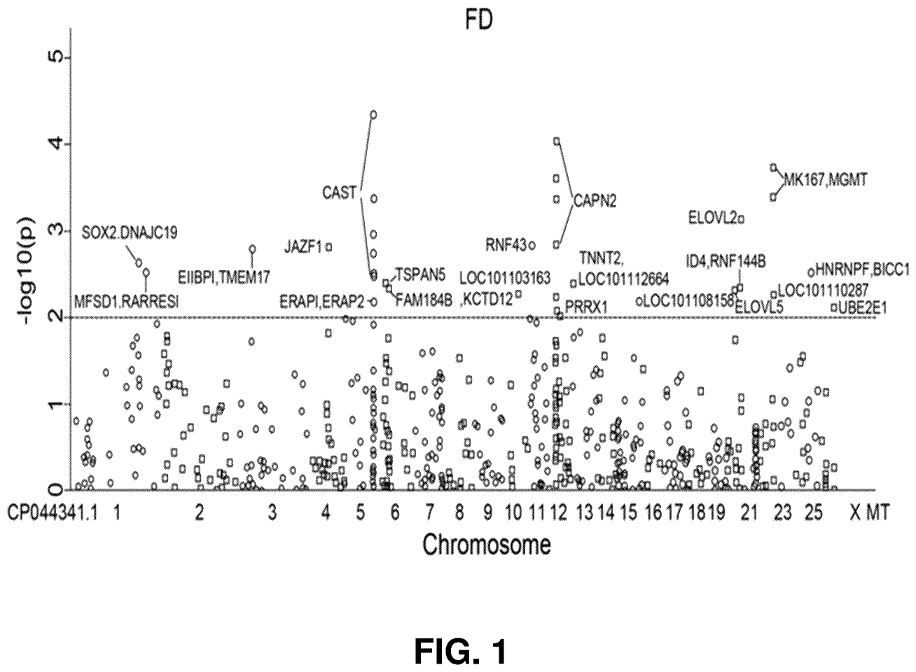

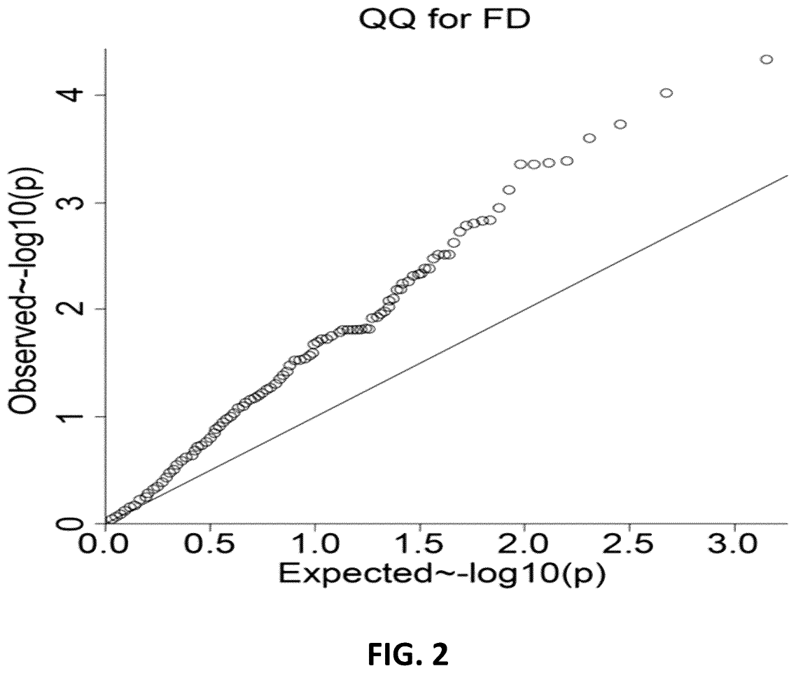

According to significant differences in the wool fiber diameter trait among the four representative fine wool sheep breeds in China (Chinese Merino sheep, Alpine Merino sheep, Aohan fine wool sheep, and Qinghai fine wool sheep), a self-written Perl script was used for marking and quality control, and the loci with an allele frequency of less than 0.05, a deletion rate of greater than 20%, or a heterozygous proportion of greater than 80% and non-biallelic loci were removed. Then, with the help of five-part population analysis, including construction of a phylogenetic tree completed by MEGA-X software, analysis of a population structure completed by Admixture software (v1.3), PCA analysis and genetic relationship analysis completed by gcta software (v1.92.2), and attenuation analysis completed by HaploviewLD software, the genetic diversity of materials and whether there are large differences among genetic backgrounds of the materials could be comprehensively evaluated to reveal a genetic similarity of non-lineage or unclear population materials and a selection degree for each subpopulation and the overall materials, thereby determining a model for adjusting GWAS. A Manhattan plot (FIG. 1) and a Q-Q plot (FIG. 2) were acquired through GLM to show mapped SNP locis associated with a wool fiber diameter of fine wool sheep and candidate genes, and significant results were screened out with a threshold of 0.01 to determine the following 30 candidate genes or markers that had definitive functions and were associated with a wool fiber diameter of fine wool sheep: SOX2, DNAJC19, MFSD1, RARRES1, EHBP1, TMEM17, JAZF1, CAST, ERAP1, ERAP2, TSPAN5, FAM184B, LOC101103163, KCTD12, RNF43, CAPN2, PRRX1, TNNT2, LOC101112664, LOC101108158, ELOVL5, ID4, RNF144B, ELOVL2, MKI67, MGMT LOC101110287, HNRNPF, BICC1, and UBE2E1.

### 3. Extraction of SNP Locis Corresponding to Functional Gene

The following unified expression of a GWAS model was adopted: y=Xα+Qβ+Kμ+e, where y represents a phenotype vector; X represents a genotype matrix; α represents a genotype effect vector; Q represents a fixed effect matrix (which can be population structure/gender/place/session information); β represents a fixed effect vector; K represents a random effect matrix, which mainly refers to a genetic relationship matrix; represents a random effect vector; and e represents a residual vector. It was determined whether α was 0 for each SNP locus. A probability p that α is 0 was used to measure a degree of association between a marker genotype and a phenotype. The smaller the p-value, the smaller the probability that α is 0 and the more likely the marker is associated with the trait. Thus, SNP locis corresponding to functional regions of the candidate genes determined in step 2 were screened to obtain the following 30 functional genes or markers associated with a wool fiber diameter trait: SOX2, DNAJC19, MFSD1, RARRES1, EHBP1, TMEM17, JAZF1, CAST ERAP1, ERAP2, TSPAN5, FAM184B, LOC101103163, KCTD12, RNF43, CAPN2, PRRX1, TNNT2, LOC101112664, LOC101108158, ELOVL5, ID4, RNF144B, ELOVL2, MKI67, MGMT LOC101110287, HNRNPF BICC1, and UBE2E1, and a combination of only 33 SNP locis.

Physical information of the combination of 33 SNP locis was specifically shown in Table 2 below.

TABLE 2
Physical information of the combination of SNP locis associated
with a wool fiber diameter trait of fine wool sheep

| number | Mark | Chr | Position | ref | mut |
|---|---|---|---|---|---|
| SNP00001 | 1_203825947 | 1 | 203825947 | G | A |
| SNP00002 | 1_226733906 | 1 | 226733906 | C | T |
| SNP00003 | 3_45470146 | 3 | 45470146 | A | C |
| SNP00004 | 4_68142771 | 4 | 68142771 | C | G |
| SNP00005 | 5_93335425 | 5 | 93335425 | C | T |
| SNP00006 | 5_93344882 | 5 | 93344882 | C | T |
| SNP00007 | 5_93387255 | 5 | 93387255 | C | T |
| SNP00008 | 5_93391985 | 5 | 93391985 | G | A |
| SNP00009 | 5_93392877 | 5 | 93392877 | C | T |
| SNP00010 | 5_93393426 | 5 | 93393426 | A | G |
| SNP00011 | 5_93507537 | 5 | 93507537 | A | C |
| SNP00012 | 6_25952072 | 6 | 25952072 | T | A |
| SNP00013 | 6_37126564 | 6 | 37126564 | T | C |
| SNP00014 | 10_51739659 | 10 | 51739659 | G | A |
| SNP00015 | 11_8917643 | 11 | 8917643 | C | A |
| SNP00016 | 12_25119445 | 12 | 25119445 | G | A |
| SNP00017 | 12_25120732 | 12 | 25120732 | C | G |
| SNP00018 | 12_25135944 | 12 | 25135944 | A | G |
| SNP00019 | 12_25149517 | 12 | 25149517 | C | A |
| SNP00020 | 12_25152554 | 12 | 25152554 | T | A |
| SNP00021 | 12_25154575 | 12 | 25154575 | C | T |
| SNP00022 | 12_25155325 | 12 | 25155325 | T | C |
| SNP00023 | 12_36292909 | 12 | 36292909 | G | A |
| SNP00024 | 12_78576808 | 12 | 78576808 | T | C |
| SNP00025 | 15_68745093 | 15 | 68745093 | C | T |
| SNP00026 | 20_25185724 | 20 | 25185724 | C | T |
| SNP00027 | 20_37863763 | 20 | 37863763 | C | T |
| SNP00028 | 20_44119346 | 20 | 44119346 | G | A |
| SNP00029 | 22_46988971 | 22 | 46988971 | G | A |
| SNP00030 | 22_47002481 | 22 | 47002481 | A | C |
| SNP00031 | 22_50203275 | 22 | 50203275 | G | A |
| SNP00032 | 25_13791395 | 25 | 13791395 | T | C |
| SNP00033 | 26_40609491 | 26 | 40609491 | C | T |

### Example 2 Panel Preparation of SNP Locis Associated with a Woo Fiber Diameter of Fine Wool Sheep

Based on the combination of SNP locis obtained in Example 1, the panel preparation of SNP locis associated with a wool fiber diameter was entrusted by the present disclosure to MolBreeding Biotech Ltd. A multiplex PCR panel mix and a multiplex PCR amplification enzyme system were added to DNA with a qualified quantitative quality inspection result, and a resulting mixture was placed on a PCR instrument to run. A PCR product was purified with carboxyl magnetic beads, and then added to a high-fidelity PCR system with Barcode-carrying sequencing primers to allow PCR amplification. Different Barcodes were adopted to distinguish different samples. After purification and amplification of amplification products with the carboxyl magnetic beads, the multiplex PCR capture and library construction were completed. Those skilled in the art can design primers by a conventional method according to sequence information of each locus in the combination of SNP locis associated with the diameter of wool fiber trait of fine wool sheep provided by the present disclosure without creative efforts. Moreover, the panel preparation is also based on the combination of SNP locis associated with the diameter of wool fiber of fine wool sheep provided by the present disclosure.

### Example 3 Detection of the Wool Fiber Diameter Trait in 437 Fine Wool Sheep Individuals

Based on the SNP locis obtained in Example 1 and the penal preparation in Example 2, the fine wool sheep individuals were tested. In an embodiment of the present disclosure, the wool fiber diameter trait of each individual was detected by GenoBaits (a targeted gene capture technical solution based on liquid-phase probe hybridization) independently developed by MolBreeding Biotech Ltd. A working principle of this technology is as follows: based on the complementary combination of a target probe with a target sequence, point-directed capture was conducted, and captured target sequences each were subjected to elution, target amplification, library construction, and sequencing to finally obtain genotypes of target SNP locis. Under cost-effective conditions, this technology is equivalent to a high-density solid-phase chip in terms of a detection density and throughput. Result values of target samples were obtained by this technique. Detection results of polymorphisms associated with the wool fiber diameter trait of fine wool sheep were shown in Table 3.

TABLE 3
Polymorphisms associated with the wool fiber diameter of fine wool sheep

| No. | Locus | Genotype | | |
|---|---|---|---|---|
| SNP00001 | 1_203825947 | AA | GA | GG |
| | | 17.9710 ± 1.3935b | 17.7940 ± 1.4706b | 17.3160 ± 1.7044a |
| SNP00002 | 1_226733906 | CC | CT | TT |
| | | 17.9220 ± 1.5094 | 17.4310 ± 1.5830 | — |
| SNP00003 | 3_45470146 | AA | AC | CC |
| | | 17.6840 ± 1.5047ab | 17.3750 ± 1.5511a | 17.9020 ± 1.5628b |
| SNP00004 | 4_68142771 | CC | CG | GG |
| | | 18.6470 ± 1.4917b | 17.8620 ± 1.4821a | 17.4730 ± 1.5815a |
| SNP00005 | 5_93335425 | CC | CT | TT |
| | | 17.8270 ± 1.5482b | 17.2920 ± 1.5330ab | 16.7830 ± 1.6453a |
| SNP00006 | 5_93344882 | CC | CT | TT |
| | | 17.8830 ± 1.6357b | 17.2520 ± 1.3145ab | 16.9390 ± 1.5370a |
| SNP00007 | 5_93387255 | CC | CT | TT |
| | | 17.4570 ± 1.4841a | 17.7440 ± 1.5872a | 18.3260 ± 1.8384b |
| SNP00008 | 5_93391985 | AA | GA | GG |
| | | 17.4010 ± 1.4389a | 17.7400 ± 1.5864ab | 18.0370 ± 1.7997b |
| SNP00009 | 5_93392877 | CC | CT | TT |
| | | 17.4010 ± 1.4389a | 17.7440 ± 1.5833ab | 18.0280 ± 1.8179b |
| SNP00010 | 5_93393426 | AA | AG | GG |
| | | 17.3180 ± 1.3961a | 17.7020 ± 1.5631ab | 17.9860 ± 1.7477b |
| SNP00011 | 5_93507537 | AA | AC | CC |
| | | 17.4010 ± 1.6456a | 17.7180 ± 1.4536ab | 18.0460 ± 1.6478b |
| SNP00012 | 6_25952072 | AA | TA | TT |
| | | 18.4040 ± 1.6079b | 17.8150 ± 1.4323a | 17.4620 ± 1.6104a |
| SNP00013 | 6_37126564 | CC | TC | TT |
| | | 18.2000 ± 1.8385 | 18.1450 ± 1.4062 | 17.5460 ± 1.5821 |
| SNP00014 | 10_51739659 | AA | GA | GG |
| | | — | 16.9000 ± 1.6378 | 17.7320 ± 1.5384 |
| SNP00015 | 11_8917643 | AA | CA | CC |
| | | 17.5340 ± 1.5329a | 17.8210 ± 1.6173a | 18.9140 ± 1.5181b |
| SNP00016 | 12_25119445 | AA | GA | GG |
| | | 16.7780 ± 1.5705a | 17.3130 ± 1.5282ab | 17.8930 ± 1.5350b |
| SNP00017 | 12_25120732 | CC | CG | GG |
| | | 18.4800 ± 1.4148b | 18.0520 ± 1.6674ab | 17.5440 ± 1.6389a |
| SNP00018 | 12_25135944 | AA | AG | GG |
| | | 17.1650 ± 1.4247a | 17.6730 ± 1.5623b | 18.1640 ± 1.5925c |
| SNP00019 | 12_2514951 | AA | CA | CC |
| | 7 | 17.5210 ± 1.6655a | 18.0320 ± 1.6134ab | 18.4080 ± 1.2718b |
| SNP00020 | 12_25152554 | AA | TA | TT |
| | | 17.0890 ± 1.3954a | 17.3690 ± 1.5390ab | 17.8790 ± 1.5683b |
| SNP00021 | 12_25154575 | CC | CT | TT |
| | | 18.4490 ± 1.1706a | 17.7380 ± 1.5650b | 17.4150 ± 1.5943b |
| SNP00022 | 12_25155325 | CC | TC | TT |
| | | 17.4150 ± 1.5843a | 17.7380 ± 1.5650a | 18.4490 ± 1.1706b |
| SNP00023 | 12_36292909 | AA | GA | GG |
| | | 17.2040 ± 1.3664a | 17.4730 ± 1.7064ab | 17.8770 ± 1.4650b |
| SNP00024 | 12_78576808 | CC | TC | TT |
| | | 17.9250 ± 1.6595b | 17.4990 ± 1.3902a | 17.3430 ± 1.7329a |
| SNP00025 | 15_68745093 | CC | CT | TT |
| | | 17.5560 ± 1.5702 | 18.3380 ± 1.4159 | 18.1670 ± 1.2423 |
| SNP00026 | 20_25185724 | CC | CT | TT |
| | | 19.0250 ± 1.3745b | 17.7140 ± 1.6412a | 17.6040 ± 1.5455a |
| SNP00027 | 20_37863763 | CC | CT | TT |
| | | 17.5260 ± 1.5450 | 18.2220 ± 1.5786 | 17.1000 ± 1.2728 |
| SNP00028 | 20_44119346 | AA | GA | GG |
| | | 17.0860 ± 1.3328a | 17.6040 ± 1.7669b | 18.0600 ± 1.5081b |
| SNP00029 | 22_46988971 | AA | GA | GG |
| | | 17.0790 ± 1.7200a | 17.3050 ± 1.5401a | 17.9280 ± 1.5163b |
| SNP00030 | 22_47002481 | AA | AC | CC |
| | | 17.8940 ± 1.4956b | 17.2520 ± 1.5973ab | 16.6460 ± 1.5967a |
| SNP00031 | 22_50203275 | AA | GA | GG |
| | | 17.5580 ± 1.5473a | 18.0530 ± 1.6216ab | 19.5500 ± 0.0707b |
| SNP00032 | 25_13791395 | CC | TC | TT |
| | | 17.1490 ± 1.3930a | 17.8330 ± 1.7487b | 17.8530 ± 1.5877b |
| SNP00033 | 26_40609491 | CC | CT | TT |
| | | 17.6820 ± 1.5775 | 17.1800 ± 1.2519 | 22.2000 ± 0.0000 |

Analysis results of correlation between different genotypes and wool fiber diameter of fine wool sheep were shown in Table 4.

TABLE 4
Analysis results of correlation between different genotypes
and wool fiber diameter of fine wool sheep

| No. | Locus | Genotype frequency | | | Gene | | χ² | P | PIC | Ne | He |
|---|---|---|---|---|---|---|---|---|---|---|---|
| | | GG | GA | AA | G | A | | | | | |
| SNP00001 | 1_203825947 | 0.372( | 0.482( | 0.147 | 0.6 | 0.3 | 0.0 | 0.7 | 0.3 | 0.4 | 1.9 |
| | | CC | CT | TT | C | T | | | | | |
| SNP00002 | 1_226733906 | 0.435( | 0.565( | 0.000 | 0.7 | 0.2 | 63. | 0.0 | 0.3 | 0.4 | 1.6 |
| | | CC | AC | AA | C | A | | | | | |
| SNP00003 | 3_45470146 | 0.460( | 0.445( | 0.095 | 0.6 | 0.3 | 0.2 | 0.5 | 0.3 | 0.4 | 1.7 |
| | | GG | CG | CC | G | C | | | | | |
| SNP00004 | 4_68142771 | 0.650( | 0.303( | 0.046 | 0.8 | 0.1 | 0.8 | 0.3 | 0.2 | 0.3 | 1.4 |
| | | CC | CT | TT | C | T | | | | | |
| SNP00005 | 5_93335425 | 0.689( | 0.281( | 0.029 | 0.8 | 0.1 | 0.0 | 0.9 | 0.2 | 0.2 | 1.3 |
| | | CC | CT | TT | C | T | | | | | |
| SNP00006 | 5_93344882 | 0.650( | 0.306( | 0.044 | 0.8 | 0.1 | 0.4 | 0.5 | 0.2 | 0.3 | 1.4 |
| | | CC | CT | TT | C | T | | | | | |
| SNP00007 | 5_93387255 | 0.491( | 0.435( | 0.073 | 0.7 | 0.2 | 1.2 | 0.2 | 0.3 | 0.4 | 1.7 |
| | | AA | GA | GG | A | G | | | | | |
| SNP00008 | 5_93391985 | 0.394( | 0.482( | 0.125 | 0.6 | 0.3 | 0.6 | 0.4 | 0.3 | 0.4 | 1.8 |
| | | CC | CT | TT | C | T | | | | | |
| SNP00009 | 5_93392877 | 0.394( | 0.484( | 0.122 | 0.6 | 0.3 | 0.8 | 0.3 | 0.3 | 0.4 | 1.8 |
| | | AA | AG | GG | A | G | | | | | |
| SNP00010 | 5_93393426 | 0.303( | 0.491( | 0.205 | 0.5 | 0.4 | 0.0 | 0.8 | 0.3 | 0.4 | 1.9 |
| | | AA | AC | CC | A | C | | | | | |
| SNP00011 | 5_93507537 | 0.386( | 0.472( | 0.142 | 0.6 | 0.3 | 0.0 | 0.9 | 0.3 | 0.4 | 1.8 |
| | | TT | TA | AA | T | A | | | | | |
| SNP00012 | 6_25952072 | 0.594( | 0.345( | 0.061 | 0.7 | 0.2 | 0.5 | 0.4 | 0.2 | 0.3 | 1.5 |
| | | TT | TC | CC | T | C | | | | | |
| SNP0 | 6_3712 | 0.839( | 0.156( | 0.005 | 0.9 | 0.0 | 0.2 | 0.5 | 0.1 | 0.1 | 1.1 |
| | | GG | GA | AA | G | A | | | | | |
| SNP00014 | 10_51739659 | 0.892( | 0.108( | 0.000 | 0.9 | 0.0 | 1.3 | 0.2 | 0.0 | 0.1 | 1.1 |
| | | AA | CA | CC | A | C | | | | | |
| SNP00015 | 11_8917643 | 0.687( | 0.293( | 0.020 | 0.8 | 0.1 | 1.3 | 0.2 | 0.2 | 0.2 | 1.3 |
| | | GG | GA | AA | G | A | | | | | |
| SNP00016 | 12_25119445 | 0.626( | 0.318( | 0.056 | 0.7 | 0.2 | 1.4 | 0.2 | 0.2 | 0.3 | 1.5 |
| | | GG | CG | CC | G | C | | | | | |
| SNP00017 | 12_25120732 | 0.719( | 0.257( | 0.024 | 0.8 | 0.1 | 0.0 | 0.8 | 0.2 | 0.2 | 1.3 |
| | | AA | AG | GG | A | G | | | | | |
| SNP00018 | 12_25135944 | 0.271( | 0.509( | 0.220 | 0.5 | 0.4 | 0.1 | 0.6 | 0.3 | 0.4 | 1.9 |
| | | AA | CA | CC | A | C | | | | | |
| SNP00019 | 12_25149517 | 0.677( | 0.291( | 0.032 | 0.8 | 0.1 | 0.0 | 0.9 | 0.2 | 0.2 | 1.4 |
| | | TT | TA | AA | T | A | | | | | |
| SNP00020 | 12_25152554 | 0.575( | 0.359( | 0.066 | 0.7 | 0.2 | 0.3 | 0.5 | 0.3 | 0.3 | 1.5 |
| | | TT | CT | CC | T | C | | | | | |
| SNP00021 | 12_25154575 | 0.484( | 0.425( | 0.090 | 0.6 | 0.3 | 0.0 | 0.8 | 0.3 | 0.4 | 1.7 |
| | | CC | TC | TT | C | T | | | | | |
| SNP00022 | 12_25155325 | 0.484( | 0.425( | 0.090 | 0.6 | 0.3 | 0.0 | 0.8 | 0.3 | 0.4 | 1.7 |
| | | GG | GA | AA | G | A | | | | | |
| SNP00023 | 12_36292909 | 0.496( | 0.389( | 0.115 | 0.6 | 0.3 | 3.3 | 0.0 | 0.3 | 0.4 | 1.7 |
| | | CC | TC | TT | C | T | | | | | |
| SNP00024 | 12_78576808 | 0.401( | 0.447( | 0.152 | 0.6 | 0.3 | 0.8 | 0.3 | 0.3 | 0.4 | 1.8 |
| | | CC | CT | TT | C | T | | | | | |
| SNP00025 | 15_68745093 | 0.888( | 0.105( | 0.007 | 0.9 | 0.0 | 1.8 | 0.1 | 0.1 | 0.1 | 1.1 |
| | | TT | CT | CC | T | C | | | | | |
| SNP00026 | 20_25185724 | 0.763( | 0.225( | 0.012 | 0.8 | 0.1 | 0.3 | 0.5 | 0.1 | 0.2 | 1.2 |
| | | CC | CT | TT | C | T | | | | | |
| SNP00027 | 20_37863763 | 0.824( | 0.171( | 0.005 | 0.9 | 0.0 | 0.6 | 0.4 | 0.1 | 0.1 | 1.1 |
| | | GG | GA | AA | G | A | | | | | |
| SNP00028 | 20_44119346 | 0.342( | 0.531( | 0.127 | 0.6 | 0.3 | 5.1 | 0.0 | 0.3 | 0.4 | 1.9 |
| | | GG | GA | AA | G | A | | | | | |
| SNP00029 | 22_46988971 | 0.562( | 0.379( | 0.059 | 0.7 | 0.2 | 0.0 | 0.7 | 0.3 | 0.3 | 1.5 |
| | | AA | AC | CC | A | C | | | | | |
| SNP00030 | 22_47002481 | 0.636( | 0.333( | 0.032 | 0.8 | 0.1 | 0.8 | 0.3 | 0.2 | 0.3 | 1.4 |
| | | AA | GA | GG | A | G | | | | | |
| SNP00031 | 22_50203275 | 0.839( | 0.156( | 0.005 | 0.9 | 0.0 | 0.2 | 0.5 | 0.1 | 0.1 | 1.1 |
| | | TT | TC | CC | T | C | | | | | |
| SNP00032 | 25_13791395 | 0.274( | 0.521( | 0.205 | 0.5 | 0.4 | 0.8 | 0.3 | 0.3 | 0.4 | 1.9 |
| | | CC | CT | TT | C | T | | | | | |
| SNP00033 | 26_40609491 | 0.897( | 0.100( | 0.002 | 0.9 | 0.0 | 0.0 | 0.8 | 0.0 | 0.1 | 1.1 |

The above results show that a wool fiber diameter of fine wool sheep can be analyzed by detecting genotypes for the combination of 33 SNP locis associated with a wool fiber diameter of fine wool sheep provided in the present disclosure, where the 33 SNP locis are located at the following positions, respectively: position 203825947 of chr 1, with a deoxynucleotide of C or A; position 226733906 of chr 1, with a deoxynucleotide of C or T; position 45470146 of chr 3, with a deoxynucleotide of A or C; position 68142771 of chr 4, with a deoxynucleotide of C or G; position 93335425 of chr 5, with a deoxynucleotide of C or T; position 93344882 of chr 5, with a deoxynucleotide of C or T; position 93387255 of chr 5, with a deoxynucleotide of C or T; position 93391985 of chr 5, with a deoxynucleotide of G or A; position 93392877 of chr 5, with a deoxynucleotide of C or T; position 93393426 of chr 5, with a deoxynucleotide of A or G; position 93507537 of chr 5, with a deoxynucleotide of A or C; position 25952072 of chr 6, with a deoxynucleotide of T or A; position 37126564 of chr 6, with a deoxynucleotide of T or C; position 51739659 of chr 10, with a deoxynucleotide of G or A; position 8917643 of chr 11, with a deoxynucleotide of C or A; position 25119445 of chr 12, with a deoxynucleotide of C or A; position 25120732 of chr 12, with a deoxynucleotide of C or G; position 25135944 of chr 12, with a deoxynucleotide of A or G; position 25149517 of chr 12, with a deoxynucleotide of C or A; position 25152554 of chr 12, with a deoxynucleotide of T or A; position 25154575 of chr 12, with a deoxynucleotide of C or T; position 25155325 of chr 12, with a deoxynucleotide of T or C; position 36292909 of chr 12, with a deoxynucleotide of G or A; position 78576808 of chr 12, with a deoxynucleotide of T or C; position 68745093 of chr 15, with a deoxynucleotide of C or T; position 25185724 of chr 20, with a deoxynucleotide of C or T; position 37863763 of chr 20, with a deoxynucleotide of C or T; position 44119346 of chr 20, with a deoxynucleotide of G or A; position 46988971 of chr 22, with a deoxynucleotide of G or A; position 47002481 of chr 22, with a deoxynucleotide of A or C; position 50203275 of chr 22, with a deoxynucleotide of G or A; position 13791395 of chr 25, with a deoxynucleotide of T or C; and position 40609491 of chr26, with a deoxynucleotide of C or T.

In the present disclosure, genotypes of target SNP locis are acquired through GenoPlexs (a targeted gene capture technical solution based on multiplex PCR) and GenoBaits (a targeted gene capture technical solution based on liquid-phase probe hybridization) technologies to allow the rapid and effective detection of a wool fiber diameter of fine wool sheep, which is of great significance for the molecular breeding of sheep and the protection and modification of germplasm resources.

Those skilled in the art can use the combination of 33 SNP locis associated with a wool fiber diameter of fine wool sheep provided by the present disclosure to prepare a SNP probes combination, a gene chip, and a kit for analyzing a wool fiber diameter of fine wool sheep. The SNP probe combination, the gene chip, and the kit can be used for analysis of a wool fiber diameter of fine wool sheep at a genome level, genetic evaluation, breed screening, or breed identification to improve the accuracy of breeding value estimation and accelerate a breeding process, and can also be used for sheep lineage reconstruction, sheep breed traceability, and germplasm resource protection and improvement. Due to the scarcity of research on the wool fiber diameter of fine wool sheep, the present disclosure is intended to accelerate a molecular breeding process of fine wool sheep, enhance the protection and improvement of species resources, and save a lot of breeding costs, thereby improving the economic benefits brought by wool of fine wool sheep.

The above are merely preferred examples to facilitate the comprehension of the present disclosure, and are not intended to limit the present disclosure. Various changes and modifications can be made by those skilled in the art to the present disclosure without departing from the idea of the present disclosure, and such changes and modifications shall also fall within the scope of the present disclosure.

What is claimed is:

1. A combination of 33 single nucleotide polymorphism (SNP) locis associated with a wool fiber diameter of fine wool sheep, wherein the combination of 33 SNP locis is determined based on alignment with an Ovis aries reference genome version 4.0 sequence, and the 33 SNP locis are located at the following positions, respectively: position 203825947 of chr 1, with a deoxynucleotide of C or A; position 226733906 of chr 1, with a deoxynucleotide of C or T; position 45470146 of chr 3, with a deoxynucleotide of A or C; position 68142771 of chr 4, with a deoxynucleotide of C or G; position 93335425 of chr 5, with a deoxynucleotide of C or T; position 93344882 of chr 5, with a deoxynucleotide of C or T; position 93387255 of chr 5, with a deoxynucleotide of C or T; position 93391985 of chr 5, with a deoxynucleotide of G or A; position 93392877 of chr 5, with a deoxynucleotide of C or T; position 93393426 of chr 5, with a deoxynucleotide of A or G; position 93507537 of chr 5, with a deoxynucleotide of A or C; position 25952072 of chr 6, with a deoxynucleotide of T or A; position 37126564 of chr 6, with a deoxynucleotide of T or C; position 51739659 of chr 10, with a deoxynucleotide of G or A; position 8917643 of chr 11, with a deoxynucleotide of C or A; position 25119445 of chr 12, with a deoxynucleotide of C or A; position 25120732 of chr 12, with a deoxynucleotide of C or G; position 25135944 of chr 12, with a deoxynucleotide of A or G; position 25149517 of chr 12, with a deoxynucleotide of C or A; position 25152554 of chr 12, with a deoxynucleotide of T or A; position 25154575 of chr 12, with a deoxynucleotide of C or T; position 25155325 of chr 12, with a deoxynucleotide of T or C; position 36292909 of chr 12, with a deoxynucleotide of G or A; position 78576808 of chr 12, with a deoxynucleotide of T or C; position 68745093 of chr 15, with a deoxynucleotide of C or T; position 25185724 of chr 20, with a deoxynucleotide of C or T; position 37863763 of chr 20, with a deoxynucleotide of C or T; position 44119346 of chr 20, with a deoxynucleotide of G or A; position 46988971 of chr 22, with a deoxynucleotide of G or A; position 47002481 of chr 22, with a deoxynucleotide of A or C; position 50203275 of chr 22, with a deoxynucleotide of G or A; position 13791395 of chr 25, with a deoxynucleotide of T or C; and position 40609491 of chr26, with a deoxynucleotide of C or T.

2. A molecular probe combination for analyzing a wool fiber diameter trait of fine wool sheep, wherein the molecular probe combination is provided to detect the combination of 33 SNP locis associated with a wool fiber diameter of fine wool sheep according to claim 1.

3. The molecular probe combination according to claim 2, wherein the molecular probe combination comprises nucleotide sequences shown in SEQ ID NO: 1 to SEQ ID NO: 156.

4. A gene chip for analyzing a wool fiber diameter trait of fine wool sheep, wherein the gene chip is loaded with the molecular probe combination for analyzing a wool fiber diameter trait of fine wool sheep according to claim 2.
